SEQUENCE LISTING (26 sequences)

SEQ ID NO: 1 - DNA, 1501 bp, synthetic construct
  misc_feature 1..1501: note = promoter pSD001
  source 1..1501: mol_type = other DNA
tccagtgtagcactaaaatctaatatcttcggctttatacttttttgttcatccgaaagcttacgaacaattctttctcctgttttattgtggatatagacaatttcgtcagtttcttggagagaagagttatttccggttttggctggccctataaacgggttcttggatttggatctagtaataaaaatgtcactgtcattctcggagctgaactttgtgttgtacgaagatgggttgttccactgttttgccagctcttcattgatgattttcttagtgggtgttcttggaggttcacgttgcctataatcttgacgttcttcttcatcactatcgatgccatcaaaattaagcgtccttattgcaggcttttgtgatttcaactgcaatccttctatctcttcatcagagctttcgaactgaatactatcactcaaaactggcgacattgcacatttccgcaaaccatttcgggaatctatgctagctcttctagacgataaagaacgaccggaaccaatacggggttgtgcaggtgggaataaatatgttggtttggattcttgacgtgaagaaggtattctagtcgatgaagtggttgataaggatatggcgtcactgagttgttttcttttcctatgttgcggtgttgggtcaggagttaattgattcacctccataactctggaatttcttgaatgtggggttttcagatgggcatctttcttgacggggttgtgagtaacggaggaacctggtgtcttgggtgtgaacggtgtttgagcctgtacgcggttacttctgggcggagtactcggagtcatgagagccattgattagaaggtgaatgagggagtcaccactctaagcaaacaaaatgaggtcgaagcaaaaaataaagtaaagtagcacttctggcaggttagatcaaagagtgacgggagatttgaagatggctggtttttccttagtcttggaagaggtttgtgtgggtatcagcgaatattccccgattaggcaaattagttgcattgaaattaacacgacatggtgatttgtggtaacaaatatctattggtggttggtgtgtgggtgtaatagtggtcgtgtcatgatgatggtgttcaggtgttgtcatagatcggtcttcagtaagagaaggaagcttggtgacgatcacagctatgatgtaatagaaattgctaagcaattgtgaggtgtgatgtattttgcagagcaattgtgcggtacaacggggtgttattgtcttcacaaggcatttattgcgaatttcgtagttgaaagaatattttagcacagggtgcttgacccctattgttgctcgctaaaccatgattgctaaatgatgacatagcaatcactttactaagattgctataaggacacctttcttagtataaatggacactcttttcccctgctaaacttcttttatttttcacacttaaacagttacaaaacacaaacacaactagaa

SEQ ID NO: 2 - DNA, 1501 bp, synthetic construct
  misc_feature 1..1501: note = promoter pSD002
  source 1..1501: mol_type = other DNA
gtgctaaaatctgaggtttacaagctgtgatgttcccctaagatctcacaatcgaacaatcgcgaagccaatgcaagttgtttaaggggaaacgactcactattcctgaaattagtattcaaaacttggtccggaagaacaatgaggcggccgttaaaatactcacgtaaacggtgtctacaagcgcattaaaatccgtttgaattcaagcaaaagccaccagaggcttatgcttggttatacccagcattgacctttggtatgagcatctgaaaaacaaccaggtgttgcaaagttaaacatccttctttgttcatatagaacccactattcatggtactccccaatcgaatttcacattctggttttgaaattacacaccacgttagcttataagatttcatataacttattgatatacggtttccattgttcgaatagttgaggttgtatgtaattcgattgaaggggccatttttgtttcctacttttcctgggagcttatccgatgcgcttcaaagctggaattgtaaatatagagaaaaagaaggatgttgttttattcttgaaagagtataattttacttctagcaactctcccacttcgcttgacttcatttatttcttgggcacataggcgtagtaatctagaccaacagataatttgccggaatgatatagcgattggaaaatgaactgaaattttttgctgtctttcaatttgacgggcagttcatcagtgaccgaccatataaatacgttgagaatgttattcttcctcgtagttgaagtggcttcataatttcagaactcaatagataaactaggatgttttaaagcaattaatgctcacaagtaaggagcgactctcttgcttttcgaatactaaaagtatcgtcccaacccagaaaaaaagacctcttaactgcaaaataaactctatatatttcttctaaaacagtttcaggttggatagtatcgcattctcatcacttctaactagtaggccatgagatatattaacgtttacttgagttctaagttctccgaattagatgcacagcacaaacaagattaggtttcacttggtacaaaatacgaacagagtttaaggtcgtaatttcatttcgttattgatccccacaatctattcttatcacagtcatcagatagtcgcgaaaaagcatgcagaaaagggggtcgtccctatctaagttgtagcattacaacaaatatgactacactcagtgtcgcaatcggtatagccaacgctgcaaaatggattctactgagaatggtatgatgatcccaggatcaatttcccaaaaattaaaaaaagtaaaataaaaagcatcagatattagggaggtggtaagattgctctgcaagcgatcacgagattttaggttttcctttatgtactatataaagcgcagattggatgccgcttttccctcctgggctatgataatatagcgaacgaaatacacgccaaaataaa

SEQ ID NO: 3 - DNA, 1500 bp, synthetic construct
  misc_feature 1..1500: note = promoter pSD003
  source 1..1500: mol_type = other DNA
tcacattcatagcatctctcgcctgcaatagcttccacgataggaatatctgtgaaagtgaacatgctatttcgatgatataagactttaagatctggcatgtttgtgttggaggttaccctggggtcaataaccctaattatctccttcactaaaaatgatgaagattcttcggattcgtttttgaacagagttaatgccatttcttcgtcaatagaaaaatcaatatctggtatctcatcttttacatattgaggatttagttttcttccctttggatagtacattatgatcaatgtattcctgtctttattgataaagtattggcattctgcttcttgtacacctttgaattgtttgtctggaagtgactgacatttttccacattgctaacggtttggcacgaattacatctaaataaaatgtcttctccggattcgtgtattaagtgatactccaatgataaatccccacctatcgaaccagaatcggcattggccacagtcacaggtaactttaggtcttgaaaaatccttctataggcttcattgacattgtcataagacttaagaccatcttctttggtcaagtcaaaagaataggcatctttcatgagaaactctcgtcctctcaacaaacctcccctaggtctcaactcatctctatatttgcgggaaatttggtacacgagaaggggtaaatctttatatgacgaacataagtcaccaactaagtttgtgatttcctcttcacaagttggcactaaacagtagtctctatccttggagtctttgaacttgaacaattcattgttgtcccatctcttagttctctcccataaatgcttggaagacaggctacttaattccatttccagcccaccagcctgatccattcttttcctaattacattttgaagctttttataggtacggagtcctaatggaagccagtgaactattcctgctgcaggctggtaaataaaccttgattgaaggagcatatcatgagtagtaaggtcctttacagaaaatagtttacttccttgaagagaagtagaataaaacctcatgttgggtctccatgaaaggttcaaaggcattgatcctttaggtacttcaggatgtttaagtcatcaaactgtccatcaaaggtagtatagtatttaccatctagatagtgatgtatgggtgtaacacaacatttaaatgttgtaaattaacattaggactgagtccggagatgctattgtcacctaaatctattagaaagcacttcagttatatcatcgatagaggtttgaagataaacctattgttgataaataaccccattacccgtttacgtagcaaggttcaaaaatttgcttagatcggagctaaaaattcgactgacttctttcgaaaatgtggattatgcaagcaacgttgctatcggaatagtatataaggtcgatctgccccattacaaattgtaaagcaacaaacatcctacgcaaa

SEQ ID NO: 4 - DNA, 1500 bp, synthetic construct
  misc_feature 1..1500: note = pSD004
  source 1..1500: mol_type = other DNA
tcagtttcacggttatgtgagctgtctccgcgtgaggcagtaacctctgtgtcatggatacaggctggtacacatttggcagtaggaacacaatctggtttagttgaaatatgggacgccacgacgtccaaatgtacaagatcaatgactgggcattcggcccgaacctcagcgctgagttggaaccgtcatgttttgagttctggttcaagagatcgcagtatcttacatcgggatgtacgtgcagcagctcactatacaagtcgcattgttgaacaccgccaagaggtttgtggcttacgttggaacgtggatgaaaacaagctggccagtggttccaatgataaccgtatgatggtatgggatgcactgcgtgtagaacagccccttatgaaagttgaagagcatactgcggctgttaaggcgttggcatggtcacctcatcaacgtggaatactggcttcgggtggaggtactgctgacagacgtatcaaggtgtggaatactttaacaggatccaagctgcacgatgttgatactggatctcaagtttgtaatctcttgtggtctcgcaattctaatgaattggtaagtactcatggatattctcgaaaccaagtcgttatttggaaatatccgcaaatgaagcaactagcatctttgactggtcatacttatcgagtcctttacctttccatgtcacctgatggaactacagtcgtaacgggggctggagacgaaactttaagattttggaactgtttcgagaagtcacgacaaagcggaggaggatcaatattactagacgcttttagtcagcttcgttaaattaccaccaaatttggtgcaaaagggcccatatggtgctacaaccaaaggaactttctaattttgataatgatgtcatttctctcatcgggatgaaaatagaagtcgaaaggatttttgtcactatttcaagccccacctgcagctggcagcatttctattgtttatgcattgtcatttatgggaaaactaagaaagttcctctccacccggactccactggtaaatatgcgatatcggaatcatgaccaacccatattttgatcctaatcatttcggttctagtctccgatcggactccgtaaaactgcggagtgaactccaacggagaatactgcagccaatctcatatttcatttgttatttgtccctcaactgtctcgataaggtcatctgtgtttgactagatgttcgtcattggcatgtcaaacaaggctagaccttacaatcatctcttacgaatgtaagtgaatgtaactatattttccttgctactttaacgaggttaaccaacccccgcacatccccacaccaccgctcttgataagcatctccgaaaatgcatgacgcgacaacttcaagcatgttgtatttactgagttttcagcctcactatcgatacctctataaatagaggcactttcgtctcttctccctccccacaagaaacca

SEQ ID NO: 5 - DNA, 1500 bp, synthetic construct
  misc_feature 1..1500: note = promoter pSD005
  source 1..1500: mol_type = other DNA
agaagtactgttatgaatcgatcgacgtgacatgttgttgatggttctgacttcttgatgtccgcgttttctgtctctcaatagtggtgttcgggggaagtatggttctaatacttaacaggtaagatggttgcaatgagcacctggtaaagcaacttgaatttcctgccctgtctccgttaagttatattcgactcaaggtccttgcttcctgtctgttctgtaaaacttccctttggtgtcttctatatcaactttaaaaacaaggtagtgtgtcgagcgatagtactgtgtctttttccctatgaaaaaaatcgcaccatccaagacttctcaccttcaacagcttcaacatcatgttcggtccttttagagctacgctggtcgatctaggaggtctgctatggaaacgtccttggagaatgtccaaaccacagaaatatagactccgcaaaagaatgcaacttgtagactccaatatcgacattatttaccagggactgactgaggagggtctgtcttgcaaagtgatagataacttgaaacaaaacttcccaaaggagcatgaagtgctccccaaaaacaagtataccgtgtttaacaagacagccaaaaactatagaaagggtgttcatttggttccaaaatggaccaagaagtctttgagagagaaccccgagttcttctaattgcacatttcttcctgttcatagattatcccacacatagttgctcacaaaaaaatcactataattttcctccaccggcagtatatcactaacacctttatctttattgtagattataatctgatctttatccttagatgtatctatcatcaaccccatgctcttgaaaagcttgagtcttaacactgtcgaatcgtagttttcttgtagatcattcgatatcactgctttttcttgctcttctaattcgttgagattctgggtcaaactagagattgaattctgaaggtgattcatgttcatctccagatctgttattgattttgctaatttaaatttttcgtgttcaagctcttcgatactctttagggtctgttgacggtcttctgtttccaataattgcttgttgaactctttaagttcgtctctctgtttactgatacgtgacaacaaatctagctggtgatcgagtttaagtttccgtttggagctcaacagagaaagattttcattaatttggttgatagtttgcacgtccggttcgatctgaaaattctctatagtcgacctgattaaggacacagtctcttgaagatcggacattggatttatggagaagggagatcaaagcggaaccagttgcactgtttacctttccagtcgagatacttatcccacagggccctcactttccaggcagaagtcacctaggaggcgcatccctccgtttgcttccctcgcgacaaactcccctgtaaaagaaaacttcactgaatcgtacacctaatcatacgacactaacacagatata

SEQ ID NO: 6 - DNA, 1500 bp, synthetic construct
  misc_feature 1..1500: note = promoter pSD007
  source 1..1500: mol_type = other DNA
gtcctttccaaatttttggttgaaggcatcgcttaaattatgagcaggatcggtggaaataagcaggtatttcttgttaggattgtgaagggcaagctggatagatatagaagaagatgtcgtggttttaccgacaccccccttacctccaacaaagatccacttcagcgattcgtggttcacaattgatcgcaaacttggctctgcctcaatatccatggttgatgtctagttgagtggcgtttgtggtctcttgatgagttcaaggcgaaagaatatgataggaaagcatggtttgaacttttcgcgaaagaaggaatactgttccgcgagaaactccccggtgccagaaccttccattgaggttaatcggtgggaggtgttcgaatgacaatgtcagacaaggcgaacacgtcttgtgacaccagctggactaagaagattcggtatgcaccgaagaagaaggccgtgtctcaattggcaactttgcaacaaactacggaggaaaagtctcacaagcttttaaccaagttgaatcacgacgacaacgataaagaaatcctcaaccatctaacacatgaagtacaaagtagaaatgtgatcttattggacaaactagaggagctcaacaaggaactgggctggattaaagaccgaaaatgaggaaccatgagcactgggcgtttccagaaaaactgcaaccaacgatgggaaaatgataccacactactatggtcaccccacattgtgaaatttcaaaccaaaaaagatcaaccccataattccccagagggttttcccaacaattttccaacggacttgataatgagtcagatcatttgagcatattcatcttaccccttattccgtgacaatttacctattccattcaaagcatacggtatcccgtgaccttctcatggagatcattctccaccgatacagcatatacacagatatacccaactaatatcaattggaccttgatatggtcgaccttgatggtcccgtccaaccttaaaacttagtttaatgctatactttcgccttgaaccaaatctgtctccccctcaatcatctctatgcaagaaggtcaacactgattacgtgagcaacagccagcaatcgttcgagtccccgccaaaaaaggcggagttactgctccttgtgaccacaccccctgagaccacgtccctaaacgatccttgtcggttccttcgtccaattggcaattgccacgcatacgtgaatcgttattgtttcgcctaccttgcgtcattcgttccagaatgttcgacatactcctctagaacataccgtcacaccaccatcttaagttatcttcacgtgaccatgacgtacattgtagttgactaccccattctcatcattccgatgcggccaaaaatctctatataaagaccgtatcccctaatattctcttcttgttaagacattaacttagttaattcaccaattactcacttataaacaaacaaa

SEQ ID NO: 7 - DNA, 1500 bp, synthetic construct
  misc_feature 1..1500: note = promoter pSD008
  source 1..1500: mol_type = other DNA
gtttctcttggggagatacttttttcgcgtgctcctccgtgcggaacttccttctgagcttctacctctcagattagtctaatcgcatcaggaataagactgagaatgcttttaaggagaggcttgagattggctaattgcgttccgaagtactctttcaaaaggagttatacccctctcaactacgattctctaaagaattatcgtaggcatgctcaggcgcctcaaccccatcagtttgacgccactagatgggaccaacaaccagttactaatgagcaaggagtaatactcccatccgactcaattgcaaacattctgagacaaccaactctggtcatagaacggcaaatggaaatgatgaatatatttttaggatttgagcaggcgaaccgatatgttatcatggatcctacaggaagtattttgggttacatgctagaaagggatctgggcatcaccaaagctatattgagacagatctaccgtttgcatcgaccttttacagtggatgtaatggatactgcaggaaatgtattaatgacaatcaagaggccgtttagtttcatcaattcgcacatcaaagctatattaccccctttcaggaacagcgacccagacgaacatgtaattggagaatccgttcaaagctggcatccttggagacgaagatacaatctatttacagcacaaattggcgaaaaggacactgtctacgatcagttcgggtacattgacgcaccgtttctttcctttgagtttcctgtactttcagaatctaggcaaacgctaggtgctgtctctagaaacttcgtgggctttgcaagagagcttttcacagatacaggagtttacatcatccgtatggggcctgaatcttttgtagggctagaagggaactacgggaacaatgtggcccaacatgcccttacgctggaccaaagggctgtattattagccaatgccgtttcaattgactttgattacttttctaggcactcgtcacacagtggtggcttcattgggtttgaggaatagacagggtctcgtcaactcagctcctgccaccaaaccaatcattgatcaacgagcacacttttgtccacgtgagatcgctttcgcttgcagaaagagcaatgcatgaaaacggcaaacgcaaaacgagcaaaaaaacgagtaaataactacaatttcaccaccaacagggtcaaagagcttttgagacactataaaaggggccctttccccccaggttccttgaaatcctcattcaattatgttttttactcataatttgactcaattggcatcttcttctttgttcatatacagtaattgatatgacgcttagtcattattagtgttctcgactagcagtggcgaaaaaagggggagttattttctagaaccgaccgcaaactataaaagaaagctgcccctcatatacctttcgaattctttattttctgtgtttcttccctatttaacatctacacaaaa

SEQ ID NO: 8 - DNA, 940 bp, unidentified
  source 1..940: mol_type = unassigned DNA; note = Komagataella phaffii
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 9 - protein, 32 aa, synthetic construct
  REGION 1..32: note = leader peptide
  VARIANT 13: note = misc_feature - Xaa can be any naturally occurring amino acid
  VARIANT 31: note = misc_feature - Xaa can be any naturally occurring amino acid
MRLLPLLSVVTLXAASPIASVQEYTDALERXR

SEQ ID NO: 10 - protein, 31 aa, unidentified
  source 1..31: note = Fusarium solani
MRLLPLLSVVTLTAASPIASVQEYTDALEKR

SEQ ID NO: 11 - protein, 31 aa, unidentified
  source 1..31: note = Fusarium solani
MRLLPLLSVVTLAAASPIASVQEYTDALETR

SEQ ID NO: 12 - protein, 31 aa, synthetic construct
  REGION 1..31: note = variant of leader peptide
MRLLPLLSVVTLAAASPIASVQEYTDALEKR

SEQ ID NO: 13 - protein, 31 aa, synthetic construct
  REGION 1..31: note = variant of leader peptide
MRLLPLLSVVTLTAASPIASVQEYTDALETR

SEQ ID NO: 14 - protein, 87 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLEKREA

SEQ ID NO: 15 - protein, 61 aa, Saccharomyces cerevisiae
MRFPSIFTAVLFAASSALAAPVNTTTEDELEGDFDVAVLPFSASIAAKEEGVSLEKREAEA

SEQ ID NO: 16 - DNA, 93 bp, synthetic construct
  misc_feature 1..93: note = nucleic acid sequence encoding variant of leader peptide
  misc_difference 37..39: note = n is a, c, g, or t
  misc_difference 88..90: note = n is a, c, g, or t
  source 1..93: mol_type = other DNA
atgaggctgcttccactgttgtccgtcgttacattgnnngccgcttccccaatcgcctctgtccaggaatacaccgacgctttggaannnaga

SEQ ID NO: 17 - DNA, 93 bp, unidentified
  source 1..93: mol_type = unassigned DNA; note = Fusarium solani
atgaggctgcttccactgttgtccgtcgttacattgactgccgcttccccaatcgcctctgtccaggaatacaccgacgctttggaaaaaaga

SEQ ID NO: 18 - DNA, 93 bp, unidentified
  source 1..93: mol_type = unassigned DNA; note = Fusarium solani
atgaggctgcttccactgttgtccgtcgttacattggctgccgcttccccaatcgcctctgtccaggaatacaccgacgctttggaaacaaga

SEQ ID NO: 19 - DNA, 93 bp, synthetic construct
  misc_feature 1..93: note = nucleic acid encoding leader peptide variant
  source 1..93: mol_type = other DNA
atgaggctgcttccactgttgtccgtcgttacattggctgccgcttccccaatcgcctctgtccaggaatacaccgacgctttggaaaaaaga

SEQ ID NO: 20 - DNA, 93 bp, synthetic construct
  misc_feature 1..93: note = nucleic acid encoding leader peptide variant
  source 1..93: mol_type = other DNA
atgaggctgcttccactgttgtccgtcgttacattgactgccgcttccccaatcgcctctgtccaggaatacaccgacgctttggaaacaaga

SEQ ID NO: 21 - DNA, 261 bp, Saccharomyces cerevisiae
  source 1..261: mol_type = unassigned DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggct

SEQ ID NO: 22 - DNA, 171 bp, Saccharomyces cerevisiae
  source 1..171: mol_type = unassigned DNA
atgagatttccttcaatttttactgctgttttattcgcagcatcctccgcattagctgctccagtcaacactacaacagaagatgaattagaaggggatttcgatgttgctgttttgccattttccgccagcattgctgctaaagaagaaggggtatctctcgagaaaaga

SEQ ID NO: 23 - protein, 317 aa, unidentified
  source 1..317: note = Fusarium solani
AITASQLDYENFKFYIQHGAAAYCNSETASGQKITCSDNGCKGVEANNAIIVASFVGKGTGIGGYVSTDNVRKEIVLSIRGSSNIRNWLTNVDFGQSSCSYVRDCGVHTGFRNAWDEIAQRARDAVAKARTMNPSYKVIATGHSLGGAVATLGAADLRSKGTAVDIFTFGAPRVGNAELSAFITAQAGGEFRVTHGRDPVPRLPPIVFGYRHTSPEYWLAGGASTKTDYTVNDIKVCEGAANLACNGGTLGLDIIAHLRYFQDTDACTAGGISWKRGDKAKRDEIPKRQEGMTDEELEQKLNDYVAMDKEYVESNKM

SEQ ID NO: 24 - DNA, 954 bp, unidentified
  source 1..954: mol_type = unassigned DNA; note = Fusarium solani
gccattactgcttctcaattggactacgaaaacttcaagttttacatccagcacggtgccgctgcttactgtaactccgaaactgcctctggtcaaaagatcacttgttccgacaacggttgcaaaggtgtcgaagctaacaacgctattattgtcgcctctttcgttggaaaaggtactggtattggtggttacgtttctactgataacgttagaaaggagatcgttttgtctattagaggttcttccaacattcgtaactggttgactaacgtcgacttcggacaatcctcttgttcttacgttagagattgtggagttcacactggtttcagaaatgcttgggacgagattgcccaaagagctagagacgctgtcgctaaagctagaactatgaacccatcttacaaggttatcgctactggtcactctttgggtggtgctgttgccactttgggtgctgctgatttgagatccaagggtactgccgtcgatatctttacttttggtgccccaagagttggtaacgctgagttgtccgctttcatcactgctcaggctggtggtgagttcagagttactcacggacgtgatccagttccacgtttgccacctatcgtcttcggttacagacacacctctccagagtactggttggctggtggtgcttccaccaagactgattatactgttaacgatatcaaggtttgtgaaggtgccgctaacttggcctgtaatggtggtactttgggattggatatcattgctcatttgagatacttccaagacactgacgcctgtactgctggtggtatctcctggaagagaggtgacaaagctaagagagatgagattccaaaaagacaagaaggaatgactgatgaggagttggaacaaaaactgaacgactatgtcgccatggataaggagtacgttgagtccaacaagatgtaa

SEQ ID NO: 25 - DNA, 2400 bp, Saccharomyces cerevisiae
  source 1..2400: mol_type = unassigned DNA
atggttttgccgattctaccgttaattgatgatctggcctcatggaatagtaagaaggaatacgtttcacttgttggtcaggtacttttggatggctcgagcctgagtaatgaagagattctccagttctccaaagaggaagaagttccattggtggctttgtccttgccaagtggtaaattcagcgatgatgaaatcattgccttcttgaacaacggagtttcttctctgttcattgctagccaagatgctaaaacagccgaacacttggttgaacaattgaatgtaccaaaggagcgtgttgttgtggaagagaacggtgttttctccaatcaattcatggtaaaacaaaaattctcgcaagataaaattgtgtccataaagaaattaagcaaggatatgttgaccaaagaagtgcttggtgaagtacgtacagaccgtcctgacggtttatataccaccctagttgtcgaccaatatgagcgttgtctagggttggtgtattcttcgaagaaatctatagcaaaggccatcgatttgggtcgtggcgtttattattctcgttctaggaatgaaatctggatcaagggtgaaacttctggcaatggccaaaagcttttacaaatctctactgactgtgattcggatgccttaaagtttatcgttgaacaagaaaacgttggattttgccacttggagaccatgtcttgctttggtgagttcaagcatggtttggtggggctagaatctttactaaaacaaaggctacaggacgctccagaggaatcttatactagaagactattcaacgactctgcattgttagatgccaagatcaaggaagaagctgaagaactgactgaggcaaagggtaagaaggagctttcttgggaggctgccgatttgttctactttgcactggccaaattagtggccaacgatgtttcattgaaggacgtcgagaataatctgaatatgaagcatctgaaggttacaagacggaaaggtgatgctaagccaaagtttgttggacaaccaaaggctgaagaagaaaaactgaccggtccaattcacttggacgtggtgaaggcttccgacaaagttggtgtgcagaaggctttgagcagaccaatccaaaagacttctgaaattatgcatttagtcaatccgatcatcgaaaatgttagagacaaaggtaactctgcccttttggagtacacagaaaagtttgatggtgtaaaattatccaatcctgttcttaatgctccattcccagaagaatactttgaaggtttaaccgaggaaatgaaggaagctttggacctttcaattgaaaacgtccgcaaattccatgctgctcaattgccaacagagactcttgaagttgaaacccaacctggtgtcttgtgttccagattccctcgtcctattgaaaaagttggtttgtatatccctggtggcactgccattttaccaagtactgcattaatgcttggtgttccagcacaagttgcccaatgtaaggagattgtgtttgcatctccaccaagaaaatctgatggtaaagtttcacccgaagttgtttatgtcgcagaaaaagttggcgcttccaagattgttctagctggtggtgcccaagccgttgctgctatggcttacgggacagaaactattcctaaagtggataaaatcttgggtccaggtaatcaatttgtgactgccgccaaaatgtatgttcaaaatgacactcaagctctatgttccattgatatgccagctggcccaagtgaagttttggttattgccgatgaagatgccgatgtggattttgttgcaagtgatttgctatcgcaagctgaacacggtattgactcccaagttatccttgttggtgttaacttgagcgaaaagaaaattcaagagattcaagatgctgtccacaatcaagctttacaactgccacgtgtggatattgttcgtaaatgtattgctcacagtacgatcgttctttgtgacggttacgaagaagcccttgaaatgtccaaccaatatgcaccagaacatttgattctacaaatcgccaatgctaacgattatgttaaattggttgacaatgcagggtccgtatttgtgggtgcttacactccagaatcgtgcggtgactattcaagtggtactaaccatacattaccaacctatggttacgctaggcagtacagtggtgccaacactgcaaccttccaaaagtttatcactgcccaaaacattacccctgaaggtttagaaaacatcggtagagctgttatgtgcgttgccaagaaggagggtctagacggtcacagaaacgctgtgaaaatcagaatgagtaagcttgggttgatcccaaaggatttccagtag

SEQ ID NO: 26 - DNA, 804 bp, Saccharomyces cerevisiae
  source 1..804: mol_type = unassigned DNA
atgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

(19) United States
(12) Patent Application Publication
(10) Pub. No.: US 2025/0207143 A1
(43) Pub. Date: Jun. 26, 2025

(54) RECOMBINANT YEAST CELL

(71) Applicant: BASF SE, Ludwigshafen am Rhein (DE)

(72) Inventors: Jingping Zhong, San Diego, CA (US); Xuqiu Tan, San Diego, CA (US); Melissa Ann Scranton, San Diego, CA (US); Daphne Li, San Diego, CA (US)

(73) Assignee: BASF SE, Ludwigshafen am Rhein (DE)

(21) Appl. No.: 19/020,303

(22) Filed: Jan. 14, 2025

Related U.S. Application Data
(63) Continuation of application No. 17/293,109, filed on May 12, 2021, now abandoned.
(60) Provisional application No. 62/769,169, filed on Nov. 19, 2018.

(51) Int. Cl. C12N 15/81; C12N 1/16; C12N 15/113; C12P 21/02; C12R 1/85

(52) U.S. Cl. CPC C12N 15/81; C12N 1/16; C12N 15/113; C12P 21/02; C12R 2001/85

(57) ABSTRACT

The present invention relates to a recombinant yeast cell for high yield protein expression. The invention further relates to cell culture involving the recombinant yeast cell, a method for preparing protein involving culturing the recombinant yeast cell and a use of the recombinant yeast cell.

### CROSS-REFERENCE TO RELATED APPLICATIONS

This application is a Continuation of U.S. application Ser. No. 17/293,109, filed on May 12, 2021, which is a 371 National Stage Entry of PCT International Application No. PCT/US2019/061929, filed on Nov. 18, 2019, which claims the benefit of priority to U.S. Application No. 62/769,169, filed on Nov. 19, 2018, the contents of which are incorporated herein by reference in their entirety.

### SEQUENCE LISTING

This application includes a nucleotide and amino acid sequence listing in computer readable form (CRF) as an XML file. The sequence listing is identified below and is hereby incorporated by reference into the specification of this application in its entirety and for all purposes.

| | Date of | Size |
|---|---|---|
| File Name | Creation | (bytes) |
| 180262US03_BSE201225USCON_SL.xml | Jan. 21, 2025 | 48,402 |
| | | bytes |

### FIELD OF THE INVENTION

The present invention relates to a recombinant yeast cell for high yield protein expression. The invention further relates to cell culture involving the recombinant yeast cell, a method for preparing protein involving culturing the recombinant yeast cell and a use of the recombinant yeast cell.

### BACKGROUND OF THE INVENTION

Komagataella phaffii (formerly designated as Pichia pastoris) is a single-celled microorganism that is easy to manipulate and culture. K. phaffii is a eukaryote capable of many of the post-translational modifications performed by higher eukaryotic cells such as proteolytic processing, folding, disulfide bond formation and glycosylation. Thus, the K. phaffii system is preferred as an expression host cell over bacterial systems, which are not capable of performing the same post-translation modifications as eukaryotic cells. Further, in bacterial systems proteins may be lost, if they are produced in inactive inclusion bodies. The K. phaffii system has been shown to give higher expression levels of protein than many bacterial systems. Hence, foreign proteins requiring post-translational modifications may be produced as biologically active molecules in K. phaffii and K. phaffii is already used for the production of a wide variety of recombinant proteins.

Expression strains disclosed in the prior art include CBS7435 (Küberl et al. (2011) J Biotechnol 154:312-320;), GS115 (DeSchutter et al. (2009) Nat Biotechnol 27:561-566) and DSMZ 70382 (Mattanovich at al. (2009) Microb Cell Factories 8:29).

Nevertheless, there is a need for optimized recombinant yeast cells allowing high yield protein expression and efficient protein recovery.

### SUMMARY OF THE INVENTION

The present inventors have developed a recombinant yeast cell that is capable of high yield protein expression due to the specific combination of promoters and leader peptides.

In particular, the present invention relates to a recombinant yeast cell comprising at least a first expression cassette which comprises
(a) a promoter comprising a nucleic acid sequence according to any one of SEQ ID Nos. 1 to 8 or a functional fragment thereof or a nucleic acid sequence which is at least 80% identical to the nucleic acid sequence according to any one of SEQ ID Nos. 1 to 8, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding a recombinant protein.

The recombinant yeast cell may further comprise a second expression cassette which comprises
(a) a promoter comprising a nucleic acid sequence according to any one of SEQ ID Nos. 1 to 8 or a functional fragment thereof or a nucleic acid sequence which is at least 80% identical to the nucleic acid sequence according to any one of SEQ ID Nos. 1 to 8, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding said recombinant protein.

The recombinant yeast cell may be deficient for at least one marker gene and may comprise a recombinant nucleic acid sequence encoding said marker.

In one embodiment the marker gene is an auxotrophic marker gene which may be selected from the group consisting of ura3, his4, ade2, arg4, ade1, ura5, met2, lys2, pro3 and tyr1.

In one embodiment, the marker gene is an antibiotic resistance marker gene which may be selected from the group consisting of zeocin resistance gene, kanamycin resistance gene, neomycin resistance gene, G418 resistance gene and hygromycin resistance gene.

The recombinant yeast cell may be deficient for a first marker gene and a second marker gene and may comprise a recombinant nucleic acid sequence encoding said first marker and a recombinant nucleic acid sequence encoding said second marker.

In one embodiment, the first and optionally the second expression cassette is stably integrated into the genome of the yeast cell.

The first and the second marker genes may be independently selected from an auxotrophic marker gene which may be independently selected from the group consisting of ura3, his4, ade 2, arg4, ade 1, ura5, met2, lys2, pro3 and tyr1.

The first and the second marker genes may be independently selected from an antibiotic resistance marker gene which may be independently selected from the group consisting of zeocin resistance gene, kanamycin resistance gene, neomycin resistance gene, G418 resistance gene and hygromycin resistance gene.

The first auxotrophic marker gene may be his4 and the second auxotrophic marker gene may be ura3.

In one embodiment, the deficiency of ura3 is due to a deletion of part or the whole of the ura3 gene.

In one embodiment, the nucleic acid sequence encoding the first auxotrophic marker is selected from the group consisting of:
(a) the nucleic acid sequence according to SEQ ID No. 25;
(b) a nucleic acid sequence which is at least 65% identical to the nucleic acid sequence according to SEQ ID No. 25;
(c) a nucleic acid sequence encoding the polypeptide according to SEQ ID No. 27; and
(d) a nucleic acid sequence encoding a polypeptide which is at least 80% identical to the polypeptide according to SEQ ID No. 27; and/or
wherein the nucleic acid sequence encoding the second auxotrophic marker is selected from the group consisting of:
(a) the nucleic acid sequence according to SEQ ID No. 26;
(b) a sequence which is at least 65% identical to the nucleic acid sequence according to SEQ ID No. 26;
(c) a nucleic acid sequence encoding the polypeptide according to SEQ ID No. 28; and
(d) a nucleic acid sequence encoding a polypeptide which is at least 80% identical to the polypeptide according to SEQ ID No. 28.

The recombinant protein may be an enzyme, a peptide, an antibody or antigen-binding fragment thereof, a protein antibiotic, a fusion protein, a vaccine or a vaccine-like protein or particle, a growth factor, a hormone or a cytokine and the enzyme may be a lipase, protease, alpha-amylase, beta-amylase, glucoamylase, xylanase, mannanase, glucanase, cellulase, or phytase.

In one embodiment the lipase is selected from:
(a) a lipase having the amino acid sequence according to SEQ ID No.23;
(b) a lipase having an amino acid sequence with at least 80% sequence identity to the amino acid sequence of SEQ ID No. 23;
(c) a lipase having one or more amino acid substitutions on positions corresponding to positions 23, 33, 82, 83, 84, 85, 160, 199, 254, 255, 256, 258, 263, 264, 265, 268, 308 or 311 of SEQ ID NO. 23;
(d) a lipase encoded by the nucleic acid sequence according to SEQ ID No. 24; and
(e) a lipase encoded by any nucleic acid sequence encoding lipase with at least 80% sequence identity to the polypeptide sequence of SEQ ID No. 23.

The yeast cell may be from a species of methylotropic yeast or may be a Komagataella phaffii cell.

The present invention further relates to a culture comprising the recombinant yeast cell as described herein.

The present invention further relates to a method for producing a recombinant protein, comprising the steps of:
(a) culturing the recombinant yeast cell as described herein in a suitable culture medium; and
(b) obtaining the recombinant protein.

The present invention further relates to the use of the recombinant yeast cell as described herein for producing a recombinant protein.

### BRIEF DESCRIPTION OF THE DRAWINGS

FIG. 1) Expression vector with K. phaffi expression cassette comprising a promoter, signal peptide, gene of interest (GOI), and terminator.

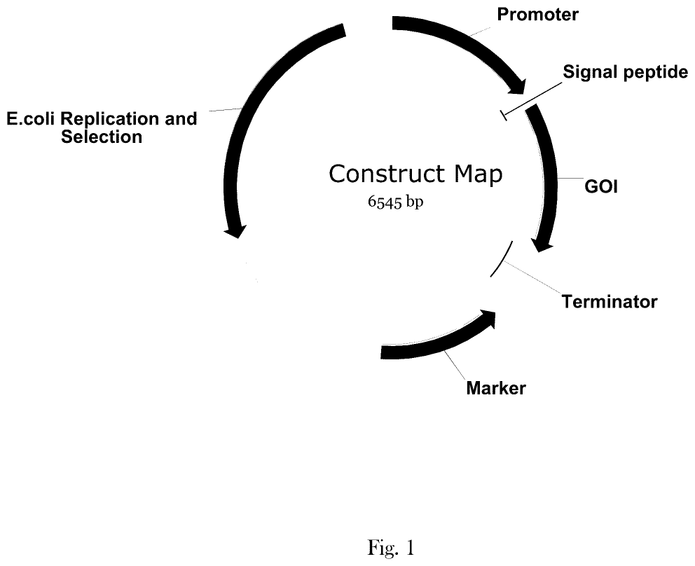

FIG. 2) Various lipases were expressed in strains transformed with different expression cassettes (1-4) in K. phaffi under fermentation conditions. Strain 1 and 2 were grown under methanol induction conditions, while strains 3 and 4 were grown under methanol-free conditions. Protein stain gels of supernatants from the final fermentation broths are shown.

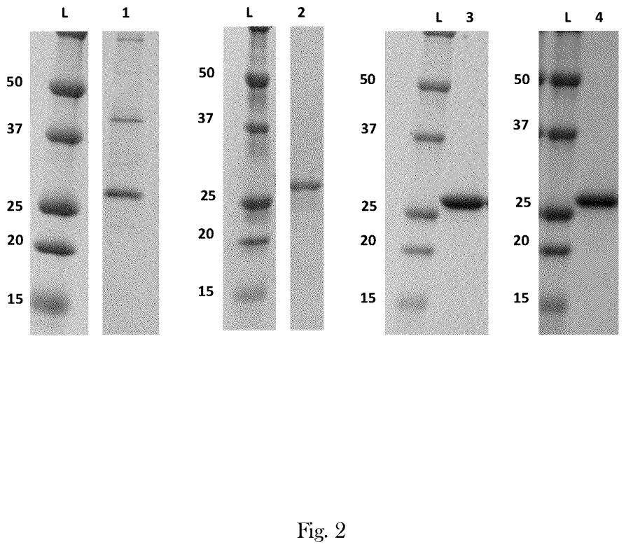

In strain 1 the lipase LIP062 as shown in Table 1 was expressed under the control of the promoter according to SEQ ID No. 8 and fused to the signal sequence according to SEQ ID No. 14.

In strain 2 the lipase LIP167 as shown in Table 1 was expressed under the control of the promoter according to SEQ ID No. 8 and fused to the signal sequence according to SEQ ID No. 15.

In strain 3 the lipase LIP134 as shown in Table 1 was expressed under the control of the promoter according to SEQ ID No. 1 and fused to the signal sequence according to SEQ ID No. 10.

In strain 4 the lipase LIP173 as shown in Table 1 was expressed under the control of the promoter according to SEQ ID No. 1 and fused to the signal sequence according to SEQ ID No. 10.

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given. Unless stated otherwise or apparent from the nature of the definition, the definitions apply to all methods and uses described herein.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay, there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The term “yeast cell” has its typical meaning. Suitable yeast cells may be selected from the genus group consisting of Pichia, Candida, Torulopsis, Arxula, Hansenula, Ogatea, Yarrowia, Kluyveromyces, Saccharomyces and Komagataella. Preferably the yeast cell is from the genus Komagataella.

In one embodiment, the yeast cell is a methylotrophic yeast cell. The term “methylotrophic yeast” as used herein includes, but is not limited to, for example, yeast species that can use reduced one-carbon compounds such as methanol or methane, and multi-carbon compounds that contain no carbon bonds, such as dimethyl ether and dimethylamine. For example, these species can use methanol as the sole carbon and energy source for cell growth. Without being limiting, methylotrophic yeast species may include the genus Methanoscacina, Methylococcus capsulatus, Hansenula polymorpha, Candida boidinii, Komagataella pastoris and Komagataella phaffii, for example. Preferably, the host cell is a Komagataella phaffii cell.

The term “recombinant yeast cell” as used herein means that the yeast cell contains at least one nucleic acid sequence which is not naturally present in the cell or which is naturally present in the yeast cell, but linked to sequences to which it is not naturally linked in the yeast cell such as a promoter to which the nucleic acid sequence encoding a protein is not naturally linked. In the context of the present invention, the recombinant yeast cell differs from the naturally occurring yeast cell in that it contains at least one expression cassette which is not present in the naturally occurring cell.

The recombinant yeast cell of the present invention has been transformed with at least a first expression cassette, i.e. a first expression cassette has been introduced into the yeast cell by the process of transformation. The presence of at least the first expression cassette in the recombinant host cell can be detected by detecting the presence of the nucleic acid sequence of the promoter by methods such as PCR or Southern Blot. Additionally or alternatively, the presence of at least the first second expression cassette in the recombinant host cell can be detected by detecting the expression of the leader peptide or the recombinant protein by methods such as Western Blot or immunofluorescence.

In one embodiment, the yeast cell which is transformed with at least the first expression cassette is deficient for at least one auxotrophic marker. In this case, the first expression cassette may comprise a nucleic acid sequence encoding said auxotrophic marker. The recombinant yeast cell may be selected using said auxotrophic marker.

Suitable methods for transforming yeast cells and in particular Komagataella phaffii cells are known to the skilled person and are described for example in Pichia Protocols, 2nd edition 2007, edited by James M. Cregg, ISBN: 978-1-58829-429-6.

The expression cassette is typically present in an expression vector which in addition to the expression cassette comprises further elements enabling its propagation and selection in bacterial cells, such as an origin of replication functional in bacterial cells and an antibiotic resistance gene functional in bacteria which enables the selection of transformed bacteria. Before the transformation the expression vector may be cut with suitable restriction enzymes to liberate the expression cassette to be transformed into the yeast cell.

In one embodiment the recombinant yeast cell is transformed with a first expression cassette and a second expression cassette. The first and the second expression cassette may be transformed separately into the yeast cell. For example, the first expression cassette may be transformed into the yeast cell and transformed cells may be selected using the first auxotrophic marker, before the second expression cassette is transformed into the yeast cell which has been transformed with the first expression cassette and transformed cells are selected using the second auxotrophic marker.

In one embodiment the first and the second expression cassette are transformed together into the yeast cell in one transformation reaction. In this case, transformed cells may be selected using the first and the second auxotrophic marker simultaneously.

The recombinant yeast cell of the present invention may be deficient for at least a first auxotrophic marker gene and optionally a second auxotrophic marker gene. Yeast cells deficient for an auxotrophic marker gene have a mutation in a gene encoding an enzyme/factor required for a certain metabolic pathway. Therefore, yeast cells deficient for certain auxotrophic marker genes are not able to synthesize a particular biochemical product and therefore the biochemical product needs to be supplemented to the growth medium. When such a yeast cell deficient for one or more auxotrophic marker genes is transformed with an expression cassette comprising a nucleic acid sequence encoding said auxotrophic marker, the ability to grow in media not supplemented with the biochemical product is restored. Hence, such auxotrophic markers can be used to select yeast cells transformed with a desired product.

Auxotrophic marker genes for use in yeast cells include, but are not limited to ura3, his4, ade2, arg4, ade1, ura5, aox1, met2, lys2, pro3 and tyr1.

Yeast cells deficient for two auxotrophic marker genes may be transformed with two expression cassettes, each comprising one auxotrophic marker gene. Transformed cells can be selected by screening for cells growing in media that has not been supplemented with the respective biochemical products. Thereby, yeast cells transformed with both expression cassettes can be selected.

In one embodiment, the first auxotrophic marker gene is his4. His4-deficient cells require the presence of histidine in their growth medium to be able to grow. Accordingly, cells transformed with a nucleic acid sequence encoding His4 are able to grow on a medium without histidine. A his4-deficient Komagataella phaffii strain GS115 is commercially available from Life Technologies™. Another his4-deficient strain CBS7435 his4 is described in Näätsaari et al. (2012) PloS One 7:e39720.

In one embodiment, the second auxotrophic marker gene is ura3. A ura3-deficient cell JC254 is described in Cregg et al. (1998) Methods Mol. Biol. 103:17-26. Ura3-deficient cells require the presence of uracil or uridine in their growth medium to be able to grow. Accordingly, cells transformed with a nucleic acid sequence encoding Ura3 are able to grow on a medium without uracil or uridine. A ura3-deficient cell JC254 is described in Cregg et al. (1998) Methods Mol. Biol. 103:17-26.

In the context of the present invention, the ura3-deficient strain may be a strain having a deletion of a part or the whole of the ura3 gene.

In one embodiment, the recombinant yeast strain is a GS115 strain which is ura3-deficient. Preferably, the recombinant yeast strain is a GS115 strain which has a partial deletion of the ura3 gene.

The terms “nucleic acid”, “nucleic acid sequence” or “nucleic acid molecule” have their usual meaning and may include, but are not limited to, for example, polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acids can be either single-stranded or double-stranded.

The term “isolated nucleic acid molecule” refers to a nucleic acid molecule that has been separated from the environment with which it is naturally associated, such as the genome.

The nucleic acid sequences used in the present invention further encompass codon-optimized sequences. A nucleic acid is codon-optimized by systematically altering codons in recombinant DNA to be expressed in a host cell other than the cell from which the nucleic acid was isolated so that the codons match the pattern of codon usage in the organism used for expression and thereby to enhance yields of an expressed protein. The codon-optimized sequence nevertheless encodes a protein with the same amino acid sequence as the native protein.

The terms “coding for” or “encoding” as used herein have their usual meaning and may include, but are not limited to, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

The term “leader peptide” as used herein refers to a peptide which directs the secretion of a protein. Proteins which are secreted from a cell have a leader peptide located at the N-terminus of the protein which is cleaved from the mature protein once the export of the nascent protein chain across the rough endoplasmic reticulum has been initiated. A leader peptide enables an expressed protein to be transported to or across the plasma membrane, thereby making it easy to separate and purify the expressed protein. Usually, leader peptides are cleaved from the protein by specialized cellular peptidases after the proteins have been transported to or across the plasma membrane.

In one embodiment, the leader peptide comprises an amino acid sequence as set forth in any one of SEQ ID Nos. 9 to 15. In one embodiment, the leader peptide is a functional variant of any one of the leader peptides according to SEQ ID Nos. 9 to 15. A functional variant of the leader peptide has essentially the same leader activity as the unmodified sequence, if the fusion of the variant leader peptide to a protein leads to essentially the same secretion of said protein into the supernatant by the recombinant host cell as the fusion of the unmodified leader peptide to said protein. Essentially the same secretion means that the amount of the protein in the supernatant of a host cell expressing the functional variant of the leader peptide is at least 50% or 60%, preferably at least 70% or 75%, more preferably at least 80% or 85% and most preferably at least 90%, 92%, 95% or 98% of the amount of the protein in the supernatant of the host cell expressing the unmodified leader peptide. In one embodiment, the leader peptide comprises the amino acid sequence as set forth in SEQ ID No. 9.

The leader peptide used in the present invention may be encoded by a nucleic acid sequence according to any one of SEQ ID NOs. 16 to 22 or a nucleic acid sequence which is at least 80% identical to the nucleic acid sequence according to any one of SEQ ID NOs. 16 to 22 and encodes a functional variant of the leader peptide according to any one of SEQ ID Nos. 9 to 15.

“Sequence Identity”, “% sequence identity”, “% identity”, “% identical” or “sequence alignment” means a comparison of a first amino acid sequence to a second amino acid sequence, or a comparison of a first nucleic acid sequence to a second nucleic acid sequence and is calculated as a percentage based on the comparison. The result of this calculation can be described as “percent identical” or “percent ID.”

Generally, a sequence alignment can be used to calculate the sequence identity by one of two different approaches. In the first approach, both mismatches at a single position and gaps at a single position are counted as non-identical positions in final sequence identity calculation. In the second approach, mismatches at a single position are counted as non-identical positions in final sequence identity calculation; however, gaps at a single position are not counted (ignored) as non-identical positions in final sequence identity calculation. In other words, in the second approach gaps are ignored in final sequence identity calculation. The difference between these two approaches, i.e. counting gaps as non-identical positions vs ignoring gaps, at a single position can lead to variability in the sequence identity value between two sequences.

A sequence identity is determined by a program, which produces an alignment, and calculates identity counting both mismatches at a single position and gaps at a single position as non-identical positions in final sequence identity calculation. For example program Needle (EMBOS), which has implemented the algorithm of Needleman and Wunsch (Needleman and Wunsch, 1970, J. Mol. Biol. 48:443-453), and which calculates sequence identity per default settings by first producing an alignment between a first sequence and a second sequence, then counting the number of identical positions over the length of the alignment, then dividing the number of identical residues by the length of an alignment, then multiplying this number by 100 to generate the % sequence identity [% sequence identity=(#of Identical residues/length of alignment)×100)].

A sequence identity can be calculated from a pairwise alignment showing both sequences over the full length, so showing the first sequence and the second sequence in their full length (“Global sequence identity”). For example, program Needle (EMBOSS) produces such alignments; % sequence identity=(#of identical residues/length of alignment)×100)].

A sequence identity can be calculated from a pairwise alignment showing only a local region of the first sequence or the second sequence (“Local Identity”). For example, program Blast (NCBI) produces such alignments; % sequence identity=(#of Identical residues/length of alignment)×100)].

The sequence alignment is preferably generated by using the algorithm of Needleman and Wunsch (J. Mol. Biol. (1979) 48, p. 443-453). Preferably, the program “NEEDLE” (The European Molecular Biology Open Software Suite (EMBOSS)) is used with the programs default parameter (gap open=10.0, gap extend=0.5 and matrix=EBLOSUM62 for proteins and matrix=EDNAFULL for nucleotides). Then, a sequence identity can be calculated from the alignment showing both sequences over the full length, so showing the first sequence and the second sequence in their full length (“Global sequence identity”). For example: % sequence identity=(#of identical residues/length of alignment)×100)].

A “variant nucleic acid sequence” as used herein refers to a nucleic acid sequence which is at least n % identical to the nucleic acid sequence of the respective parent sequence with “n” being an integer between 80 and 100. The variant nucleic acid sequences include sequences that are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical when compared to the full-length sequence of the parent nucleic acid sequence. The variant nucleic acid sequence encodes a protein having essentially the same activity as the protein encoded by the parent nucleic acid sequence.

A “variant amino acid sequence” as used herein refers to an amino acid sequence which is at least n % identical to the amino acid sequence of the respective parent sequence with “n” being an integer between 80 and 100. The variant amino acid sequences include sequences that are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical when compared to the full-length sequence of the parent amino acid sequence. The protein having a variant amino acid sequence has essentially the same activity as the parent amino acid sequence.

The term “expression cassette” refers to a nucleic acid molecule containing the coding sequence of a protein and control sequences such as e.g. a promoter in operable linkage, so that host cells transformed or transfected with these sequences are capable of producing the encoded proteins. The expression cassette may further comprise a nucleic acid sequence encoding a marker such as an auxotrophic marker. The expression cassette may be part of a vector or may be integrated into the host cell chromosome.

The expression cassette may further contain a suitable terminator sequence operably linked to the nucleic acid sequence encoding the protein. Suitable terminator sequences include, but are not limited to, the AOX1 (alcohol oxidase) terminator, the CYC1 (cytochrome c) terminator and the TEF (translation elongation factor) terminator.

In one embodiment, the recombinant yeast cell comprises one expression cassette which comprises:
(a) a promoter comprising a nucleic acid sequence according to any one of SEQ ID Nos. 1 to 8 or a nucleic acid sequence which is at least 80% identical to the sequence according to any one of SEQ ID Nos. 1 to 8, operatively linked thereto
(b) a nucleic acid sequence encoding a leader peptide comprising an amino acid sequence according to SEQ ID No. 9 or an amino acid sequence which is at least 80% identical to the sequence according to SEQ ID No. 9; and
(c) a nucleic acid sequence encoding a recombinant protein.

In one embodiment, the recombinant yeast cell comprises one expression cassette which comprises:
(a) a promoter comprising a nucleic acid sequence according to any one of SEQ ID Nos. 1 to 8 or a nucleic acid sequence which is at least 80% identical to the sequence according to any one of SEQ ID Nos. 1 to 8, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a recombinant protein.

In one embodiment, the recombinant yeast cell comprises one expression cassette which comprises:
(a) a promoter comprising a nucleic acid sequence according to any one of SEQ ID Nos. 1 to 8 or a nucleic acid sequence which is at least 80% identical to the sequence according to any one of SEQ ID Nos. 1 to 8, operatively linked thereto
(b) a nucleic acid sequence encoding a leader peptide comprising an amino acid sequence according to SEQ ID No. 11 or an amino acid sequence which is at least 80% identical to the sequence according to SEQ ID No. 11; and
(c) a nucleic acid sequence encoding a recombinant protein.

In one embodiment, the recombinant yeast cell comprises one expression cassette which comprises:
(a) a promoter comprising a nucleic acid sequence according to any one of SEQ ID Nos. 1 to 8 or a nucleic acid sequence which is at least 80% identical to the sequence according to any one of SEQ ID Nos. 1 to 8, operatively linked thereto
(b) a nucleic acid sequence encoding a leader peptide comprising an amino acid sequence according to SEQ ID No. 12 or an amino acid sequence which is at least 80% identical to the sequence according to SEQ ID No. 12; and
(c) a nucleic acid sequence encoding a recombinant protein.

In one embodiment, the recombinant yeast cell comprises one expression cassette which comprises:
(a) a promoter comprising a nucleic acid sequence according to any one of SEQ ID Nos. 1 to 8 or a nucleic acid sequence which is at least 80% identical to the sequence according to any one of SEQ ID Nos. 1 to 8, operatively linked thereto
(b) a nucleic acid sequence encoding a leader peptide comprising an amino acid sequence according to SEQ ID No. 13 or an amino acid sequence which is at least 80% identical to the sequence according to SEQ ID No. 13; and
(c) a nucleic acid sequence encoding a recombinant protein.

In one embodiment, the recombinant yeast cell comprises one expression cassette which comprises:
(a) a promoter comprising a nucleic acid sequence according to any one of SEQ ID Nos. 1 to 8 or a nucleic acid sequence which is at least 80% identical to the sequence according to any one of SEQ ID Nos. 1 to 8, operatively linked thereto
(b) a nucleic acid sequence encoding a leader peptide comprising an amino acid sequence according to SEQ ID No. 14 or an amino acid sequence which is at least 80% identical to the sequence according to SEQ ID No. 14; and
(c) a nucleic acid sequence encoding a recombinant protein.

In one embodiment, the recombinant yeast cell comprises one expression cassette which comprises:
(a) a promoter comprising a nucleic acid sequence according to any one of SEQ ID Nos. 1 to 8 or a nucleic acid sequence which is at least 80% identical to the sequence according to any one of SEQ ID Nos. 1 to 8, operatively linked thereto
(b) a nucleic acid sequence encoding a leader peptide comprising an amino acid sequence according to SEQ ID No. 15 or an amino acid sequence which is at least 80% identical to the sequence according to SEQ ID No. 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 1 or a nucleic acid sequence which is at least 80% identical to the sequence according to any one of SEQ ID No. 1, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 2 or a nucleic acid sequence which is at least 80% identical to the sequence according to any one of SEQ ID No. 2, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 3 or a nucleic acid sequence which is at least 80% identical to the sequence according to any one of SEQ ID No. 3, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 4 or a nucleic acid sequence which is at least 80% identical to the sequence according to any one of SEQ ID No. 4, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 5 or a nucleic acid sequence which is at least 80% identical to the sequence according to any one of SEQ ID No. 5, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 6 or a nucleic acid sequence which is at least 80% identical to the sequence according to any one of SEQ ID No. 6, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 7 or a nucleic acid sequence which is at least 80% identical to the sequence according to any one of SEQ ID No. 7, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to any one of SEQ ID No. 8, operatively linked thereto
(b) a nucleic acid sequence encoding a leader peptide comprising an amino acid sequence according to any one of SEQ ID Nos. 9 to 15 or an amino acid sequence which is at least 80% identical to the sequence according to any one of SEQ ID Nos. 9 to 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 14 or an amino acid sequence which is at least 80% identical to the sequence according to SEQ ID No. 14; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 15 or an amino acid sequence which is at least 80% identical to the sequence according to SEQ ID No. 15; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 1 or a nucleic acid sequence which is at least 80% identical to the sequence according to SEQ ID No. 1, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 2 or a nucleic acid sequence which is at least 80% identical to the sequence according to SEQ ID No. 2, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 3 or a nucleic acid sequence which is at least 80% identical to the sequence according to SEQ ID No. 3, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 4 or a nucleic acid sequence which is at least 80% identical to the sequence according to SEQ ID No. 4, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 6 or a nucleic acid sequence which is at least 80% identical to the sequence according to SEQ ID No. 6, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a recombinant protein.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 14 or an amino acid sequence which is at least 80% identical to the sequence according to SEQ ID No. 14; and
(c) a nucleic acid sequence encoding the lipase according to SEQ ID No. 23.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 15 or an amino acid sequence which is at least 80% identical to the sequence according to SEQ ID No. 15; and
(c) a nucleic acid sequence encoding the lipase according to SEQ ID No. 23.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 15 or an amino acid sequence which is at least 80% identical to the sequence according to SEQ ID No. 15; and
(c) a nucleic acid sequence encoding a variant of the lipase according to SEQ ID No. 23 which comprises the following amino acid substitutions in comparison to the amino acid sequence of SEQ ID No. 23: S83H, I85T and D265S and T268G.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 15 or an amino acid sequence which is at least 80% identical to the sequence according to SEQ ID No. 15; and
(c) a nucleic acid sequence encoding a variant of the lipase according to SEQ ID No. 23 which comprises the following amino acid substitutions in comparison to the amino acid sequence of SEQ ID No. 23: S83H, I85L and T268G.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding the lipase according to SEQ ID No. 23.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitution in comparison to the amino acid sequence of SEQ ID No. 23: I255A.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 15 or an amino acid sequence which is at least 80% identical to the sequence according to SEQ ID No. 15; and
(c) a nucleic acid sequence encoding a variant of the lipase according to SEQ ID No. 23 which comprises the following amino acid substitutions in comparison to the amino acid sequence of SEQ ID No. 23: S83H, I85L and D265T.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 15 or an amino acid sequence which is at least 80% identical to the sequence according to SEQ ID No. 15; and
(c) a nucleic acid sequence encoding a variant of the lipase according to SEQ ID No. 23 which comprises the following amino acid substitutions in comparison to the amino acid sequence of SEQ ID No. 23: S83H, I85L and D265A.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 15 or an amino acid sequence which is at least 80% identical to the sequence according to SEQ ID No. 15; and
(c) a nucleic acid sequence encoding a variant of the lipase according to SEQ ID No. 23 which comprises the following amino acid substitutions in comparison to the amino acid sequence of SEQ ID No. 23: S83H, I254L and D265T.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 15 or an amino acid sequence which is at least 80% identical to the sequence according to SEQ ID No. 15; and
(c) a nucleic acid sequence encoding a variant of the lipase according to SEQ ID No. 23 which comprises the following amino acid substitutions in comparison to the amino acid sequence of SEQ ID No. 23: I255A and D265S.

In one embodiment, the recombinant yeast cell comprises one expression cassette which comprises:
(a) a promoter comprising a nucleic acid sequence according to SEQ ID No. 1 or a nucleic acid sequence which is at least 80% identical to the sequence according to SEQ ID No. 1, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitution in comparison to the amino acid sequence of SEQ ID No. 23: I255A.

In one embodiment, the recombinant yeast cell comprises one expression cassette which comprises:
(a) a promoter comprising a nucleic acid sequence according to SEQ ID No. 2 or a nucleic acid sequence which is at least 80% identical to the sequence according to SEQ ID No. 2, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitution in comparison to the amino acid sequence of SEQ ID No. 23: I255A.

In one embodiment, the recombinant yeast cell comprises one expression cassette which comprises:
(a) a promoter comprising a nucleic acid sequence according to SEQ ID No. 8 or a nucleic acid sequence which is at least 80% identical to the sequence according to SEQ ID No. 8, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitutions in comparison to the amino acid sequence of SEQ ID No. 23: I255A and D265S.

In one embodiment, the recombinant yeast cell comprises one expression cassette which comprises:
(a) a promoter comprising a nucleic acid sequence according to SEQ ID No. 1 or a nucleic acid sequence which is at least 80% identical to the sequence according to SEQ ID No. 1, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitutions in comparison to the amino acid sequence of SEQ ID No. 23: I255A and D265S.

In one embodiment, the recombinant yeast cell comprises one expression cassette which comprises:
(a) a promoter comprising a nucleic acid sequence according to SEQ ID No. 1 or a nucleic acid sequence which is at least 80% identical to the sequence according to SEQ ID No. 1, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitutions in comparison to the amino acid sequence of SEQ ID No. 23: S83H, I85L and T268G.

In one embodiment, the recombinant yeast cell comprises one expression cassette which comprises:
(a) a promoter comprising a nucleic acid sequence according to SEQ ID No. 3 or a nucleic acid sequence which is at least 80% identical to the sequence according to SEQ ID No. 3, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitutions in comparison to the amino acid sequence of SEQ ID No. 23: I255A and D265S.

In one embodiment, the recombinant yeast cell comprises one expression cassette which comprises:
(a) a promoter comprising a nucleic acid sequence according to SEQ ID No. 4 or a nucleic acid sequence which is at least 80% identical to the sequence according to SEQ ID No. 4, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitutions in comparison to the amino acid sequence of SEQ ID No. 23: I255A and D265S.

In one embodiment, the recombinant yeast cell comprises one expression cassette which comprises:
(a) a promoter comprising a nucleic acid sequence according to SEQ ID No. 6 or a nucleic acid sequence which is at least 80% identical to the sequence according to SEQ ID No. 6, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitutions in comparison to the amino acid sequence of SEQ ID No. 23: I255A and D265S.

In one embodiment, the recombinant yeast cell comprises one expression cassette which comprises:
(a) a promoter comprising a nucleic acid sequence according to SEQ ID No. 6 or a nucleic acid sequence which is at least 80% identical to the sequence according to SEQ ID No. 6, operatively linked thereto
(b) a nucleic acid sequence encoding a leader peptide comprising an amino acid sequence according to SEQ ID No. 10 or an amino acid sequence which is at least 80% identical to the sequence according to SEQ ID No. 10; and
(c) a nucleic acid sequence encoding a variant of the lipase according to SEQ ID No. 23 which comprises the following amino acid substitutions in comparison to the amino acid sequence of SEQ ID No. 23: S83H, I85L and T268G.

In one embodiment, the recombinant yeast cell comprises a first and a second expression cassette. In one embodiment, the first and the second expression cassette encode the same recombinant protein, i.e. the recombinant protein encoded by the first expression cassette is the same as the recombinant protein encoded by the second expression cassette.

In one embodiment, the first expression cassette comprises a promoter sequence that is at least 80% identical to the promoter sequence according to any one of SEQ ID Nos. 1 to 8, a nucleic acid sequence encoding a recombinant protein and a nucleic acid sequence encoding a first auxotrophic marker.

In one embodiment, the first expression cassette comprises a promoter sequence that is at least 80% identical to the promoter sequence according to any one of SEQ ID Nos. 1 to 8, a nucleic acid sequence encoding a recombinant protein and a nucleic acid sequence encoding HIS4.

In one embodiment, the first expression cassette comprises a promoter sequence that is at least 80% identical to the promoter sequence according to any one of SEQ ID Nos. 1 to 8, a nucleic acid sequence encoding a recombinant protein and a nucleic acid sequence according to SEQ ID No. 25 or a nucleic acid sequence which is 80% identical thereto encoding HIS4.

In one embodiment, the first expression cassette comprises a promoter sequence according to SEQ ID No. 1, a nucleic acid sequence encoding a recombinant protein, a terminator sequence and the nucleic acid sequence according to SEQ ID No. 25 operably linked to a suitable promoter and terminator.

In one embodiment, the first expression cassette comprises a promoter sequence according to SEQ ID No. 1, a nucleic acid sequence encoding the leader peptide according to SEQ ID No. 9, a nucleic acid sequence encoding a recombinant lipase, a terminator sequence and the nucleic acid sequence according to SEQ ID No. 25 operably linked to a suitable promoter and terminator.

In one embodiment, the second expression cassette comprises a promoter sequence that is at least 80% identical to the promoter sequence according to any one of SEQ ID Nos. 1 to 8, a nucleic acid sequence encoding a recombinant protein and a nucleic acid sequence encoding a second auxotrophic marker.

In one embodiment, the second expression cassette comprises a promoter sequence that is at least 80% identical to the promoter sequence according to any one of SEQ ID Nos. 1 to 8, a nucleic acid sequence encoding a recombinant protein and a nucleic acid sequence encoding URA3.

In one embodiment, the second expression cassette comprises a promoter sequence that is at least 80% identical to the promoter sequence according to any one of SEQ ID Nos. 1 to 8, a nucleic acid sequence encoding a recombinant protein and a nucleic acid sequence according to SEQ ID No. 26 or a nucleic acid sequence which is 80% identical thereto encoding URA3.

In one embodiment, the second expression cassette comprises a promoter sequence according to SEQ ID No. 1, a nucleic acid sequence encoding a recombinant protein, a terminator sequence and the nucleic acid sequence according to SEQ ID No. 26 operably linked to a suitable promoter and terminator.

In one embodiment, the second expression cassette comprises a promoter sequence according to SEQ ID No. 1, a nucleic acid sequence encoding the leader peptide according to SEQ ID No. 9, a nucleic acid sequence encoding a recombinant lipase, a terminator sequence and the nucleic acid sequence according to SEQ ID No. 26 operably linked to a suitable promoter and terminator.

In one embodiment, the first expression cassette comprises a promoter sequence that is at least 80% identical to the promoter sequence according to any one of SEQ ID Nos. 1 to 8, a nucleic acid sequence encoding a recombinant protein and a nucleic acid sequence encoding a first auxotrophic marker and the second expression cassette comprises a promoter sequence that is at least 80% identical to the promoter sequence according to any one of SEQ ID Nos. 1 to 8, a nucleic acid sequence encoding a recombinant protein and a nucleic acid sequence encoding a second auxotrophic marker.

In one embodiment, the first expression cassette comprises a promoter sequence that is at least 80% identical to the promoter sequence according to any one of SEQ ID Nos. 1 to 8, a nucleic acid sequence encoding a recombinant protein and a nucleic acid sequence encoding HIS4 and the second expression cassette comprises a promoter sequence that is at least 80% identical to the promoter sequence according to any one of SEQ ID Nos. 1 to 8, a nucleic acid sequence encoding a recombinant protein and a nucleic acid sequence encoding URA3.

In one embodiment, the first expression cassette comprises a promoter sequence that is at least 80% identical to the promoter sequence according to any one of SEQ ID Nos. 1 to 8, a nucleic acid sequence encoding a recombinant protein and a nucleic acid sequence according to SEQ ID No. 25 or a nucleic acid sequence which is 80% identical thereto encoding HIS4 and the second expression cassette comprises a promoter sequence that is at least 80% identical to the promoter sequence according to any one of SEQ ID Nos. 1 to 8, a nucleic acid sequence encoding a recombinant protein and a nucleic acid sequence according to SEQ ID No. 26 or a nucleic acid sequence which is 80% identical thereto encoding URA3.

In one embodiment, the first expression cassette comprises a promoter sequence according to SEQ ID No. 1, a nucleic acid sequence encoding a recombinant protein, a terminator sequence and the nucleic acid sequence according to SEQ ID No. 25 operably linked to a suitable promoter and terminator and the second expression cassette comprises a promoter sequence according to SEQ ID No. 1, a nucleic acid sequence encoding a recombinant protein, a terminator sequence and the nucleic acid sequence according to SEQ ID No. 26 operably linked to a suitable promoter and terminator.

In one embodiment, the first expression cassette comprises a promoter sequence according to SEQ ID No. 1, a nucleic acid sequence encoding the leader peptide according to SEQ ID No. 9, a nucleic acid sequence encoding a recombinant lipase, a terminator sequence and the nucleic acid sequence according to SEQ ID No. 25 operably linked to a suitable promoter and terminator and the second expression cassette comprises a promoter sequence according to SEQ ID No. 1, a nucleic acid sequence encoding the leader peptide according to SEQ ID No. 9, a nucleic acid sequence encoding a recombinant lipase, a terminator sequence and the nucleic acid sequence according to SEQ ID No. 26 operably linked to a suitable promoter and terminator.

The recombinant protein expressed in the yeast cell of the invention present invention can be any protein such as any eukaryotic, prokaryotic and synthetic protein. The protein may be homologous to the host cell, i.e. it may be naturally expressed by the host cell, or it can be heterologous to the host cell, i.e. it may not be naturally expressed by the host cell. The protein can include, but is not limited to, enzymes, peptides, antibodies and antigen-binding fragments thereof and recombinant proteins. Proteins obtained by heterologous expression in K. phaffi which are already on the market include phytase, trypsin, nitrate reductase, phospholipase C, collagen, proteinase K, ecallantide, ocriplasmin, human insulin, pleactasin peptide derivative NZ2114, elastase inhibitor, recombinant cytokines and growth factors, human cystatin C, HB-EGF, interferon-alpha 2b, human serum albumin and human angiostatin.

In one embodiment the recombinant protein is an enzyme. The enzyme may be selected from the group consisting of lipase, alpha-amylase, beta-amylase, glucoamylase, protease, xylanase, glucanase, cellulase, mannanase and phytase.

In one embodiment, the recombinant protein encoded by the first and second expression cassette is a lipase. The lipase may have an amino acid sequence having at least 80% sequence identity to the amino acid sequence of SEQ ID No. 23. In one embodiment, the lipase has an amino acid sequence according to SEQ ID No. 23. In one embodiment, the lipase is encoded by a nucleic acid sequence having at least 80% sequence identity to the nucleic acid sequence of SEQ ID No. 24. In one embodiment, the lipase is encoded by the nucleic acid sequence according to SEQ ID No. 24. The protein having an amino acid sequence which is at least 80% identical to the amino acid sequence of SEQ ID No. 23 or which is encoded by a nucleic acid sequence which is at least 80% identical to the nucleic acid sequence of SEQ ID No. 24 has lipase activity. The term “lipase activity” means that the protein can cleave ester bonds in lipids. The lipase activity of a protein can be determined by incubating the protein with a suitable lipase substrate, such as PNP-octanoate, 1-olein, galactolipids, phosphatidylcholine and triacylglycerols and determining the lipase activity in comparison to a control lipase.

In one embodiment, the lipase comprises one or more amino acid insertions, deletions or substitutions in comparison to the amino acid sequence of SEQ ID No. 23. In one embodiment, the amino acid insertion, deletion or substitution in comparison to the amino acid sequence of SEQ ID No. 23 is at an amino acid residue selected from amino acid residues 23, 33, 82, 83, 84, 85, 160, 199, 254, 255, 256, 258, 263, 264, 265, 268, 308 and 311. In one embodiment, the amino acid substitution in comparison to the amino acid sequence of SEQ ID No. 23 is selected from the group consisting of: Y23A, K33N, S82T, S83D, S83H, S831, S83N, S83R, S83T, S83Y, S84S, S84N, I85A, I85C, I85F, I85H, I85L, I85M, I85P, I85S, I85T, I85V, I85Y, K160N, P199I, P199V, I254A, I254C, I254E, I254F, I254G, I254L, I254M, I254N, I254R, I254S, I2454V, I254W, I254Y, I255A, I255L, A256D, L258A, L258D; L258E, L258G, L258H, L258N, L258Q, L258R, L258S, L258T, L258V, D263G, D263K, D263P, D263R, D263S; T264A, T264D, T264G, T264I, T264L, T264N, T264S, D265A, D265G, D265K, D265L, D265N, D265S, D265T, T268A, T268G, T268K, T268L, T268N, T268S, D308A, and Y311E. In one embodiment, the lipase comprises the following amino acid substitutions in comparison to the amino acid sequence of SEQ ID No. 23: S83H, I85L and T268G. In one embodiment, the lipase comprises the following amino acid substitutions in comparison to the amino acid sequence of SEQ ID No. 23: I255A and D265S. In one embodiment, the lipase comprises the following amino acid substitutions in comparison to the amino acid sequence of SEQ ID No. 23: S83H, I254L and D265T. In one embodiment, the lipase comprises the following amino acid substitutions in comparison to the amino acid sequence of SEQ ID No. 23: S83H, I85L and D265T. In one embodiment, the lipase comprises the following amino acid substitutions in comparison to the amino acid sequence of SEQ ID No. 23: S83H, I85L and D265A. In one embodiment, the lipase comprises the following amino acid substitution in comparison to the amino acid sequence of SEQ ID No. 23: I255A. In one embodiment, the lipase comprises the following amino acid substitutions in comparison to the amino acid sequence of SEQ ID No. 23: S83H, I85T and D265S and T268G.

Further suitable lipases having one or more amino acid substitutions or insertions compared to the sequence according to SEQ ID No. 23 are shown in the following Table 1 wherein LIP062 refers to the lipase according to SEQ ID No. 23.

TABLE: 1

| Lipase | Amino Acid Residue Position Numbers | | | | | | | | | | | | | | | | | | |
|---|---|---|---|---|---|---|---|---|---|---|---|---|---|---|---|---|---|---|---|
| | 2 | 3 | 8 | 8 | 8 | 8 | 8 | 16 | 19 | 25 | 25 | 25 | 25 | 26 | 26 | 26 | 26 | 30 | 31 |
| | 3 | 3 | 2 | 3 | 4 | 4′ | 5 | 0 | 9 | 4 | 5 | 6 | 8 | 3 | 4 | 5 | 8 | 8 | 1 |
| LIP062 | Y | K | S | S | N | | I | K | P | I | I | A | L | D | T | D | T | D | Y |
| LIP182 | — | — | — | H | — | — | S | — | — | — | A | — | — | — | A | T | — | — | — |
| LIP181 | — | — | — | H | — | — | V | — | — | — | A | — | — | — | S | T | — | — | — |
| LIP180 | — | — | — | T | — | — | H | — | — | — | A | — | — | — | — | A | — | — | — |
| LIP179 | — | — | — | — | — | — | V | — | — | — | A | — | — | — | S | T | — | — | — |
| LIP178 | — | — | — | H | — | — | L | — | — | — | A | — | — | — | S | A | — | — | — |
| LIP177 | — | — | — | H | — | — | T | — | — | — | A | — | — | — | — | T | — | — | — |
| LIP176 | — | — | — | Y | — | — | A | — | — | — | A | — | — | — | — | T | — | — | — |
| LIP175 | — | — | — | T | — | — | V | — | — | — | A | — | — | — | — | S | — | — | — |
| LIP174 | — | — | — | — | — | — | — | — | — | — | A | — | — | — | — | A | — | — | — |
| LIP173 | — | — | — | — | — | — | — | — | — | — | A | — | — | — | — | S | — | — | — |
| LIP172 | — | — | — | N | — | — | L | — | — | — | A | — | — | — | N | T | — | — | — |
| LIP171 | — | — | — | — | — | — | — | — | — | — | A | — | — | — | D | T | — | — | — |
| LIP170 | — | — | — | N | — | — | L | — | — | — | A | — | — | — | — | T | — | — | — |
| LIP169 | — | — | — | N | — | — | V | — | — | L | — | — | — | — | S | T | — | — | — |
| LIP168 | — | — | — | H | — | — | — | — | — | L | — | — | — | — | A | A | — | — | — |
| LIP167 | — | — | — | H | — | — | — | — | — | L | — | — | — | — | — | T | — | — | — |
| LIP166 | — | — | — | — | — | — | V | — | — | L | — | — | — | — | D | T | — | — | — |
| LIP165 | — | — | — | Y | — | — | — | — | — | — | — | — | — | — | A | T | — | — | — |
| LIP164 | — | — | — | — | — | — | V | — | — | — | — | — | — | — | D | T | — | — | — |
| LIP163 | — | — | — | Y | — | — | A | — | — | — | — | — | — | — | A | T | — | — | — |
| LIP162 | — | — | — | N | — | — | V | — | — | — | — | — | — | — | N | T | — | — | — |
| LIP161 | — | — | — | N | — | — | — | — | — | — | — | — | — | — | D | T | — | — | — |
| LIP160 | — | — | — | H | — | — | L | — | — | — | — | — | — | — | — | T | — | — | — |
| LIP159 | — | — | — | H | — | — | A | — | — | — | — | — | — | — | A | T | — | — | — |
| LIP158 | — | — | — | T | — | — | V | — | — | — | — | — | — | — | — | T | — | — | — |
| LIP157 | — | — | — | H | — | — | L | — | — | — | — | — | — | — | — | A | — | — | — |
| LIP156 | — | — | — | H | — | — | V | — | — | — | — | — | — | — | — | A | — | — | — |
| LIP155 | — | — | — | T | — | — | A | — | — | — | — | — | — | — | — | T | — | — | — |
| LIP154 | — | — | — | H | — | — | V | — | — | — | — | — | — | — | N | T | — | — | — |
| LIP153 | — | — | — | — | — | — | V | — | — | — | — | — | — | — | — | G | — | — | — |
| LIP152 | — | — | — | H | — | — | — | — | — | — | — | — | — | — | — | A | — | — | — |
| LIP151 | — | — | — | Y | — | — | V | — | — | — | — | — | — | — | S | S | — | — | — |
| LIP150 | — | — | — | N | — | — | V | — | — | — | — | — | — | — | — | G | — | — | — |
| LIP149 | — | — | — | H | — | — | — | — | — | — | — | — | — | — | — | S | — | — | — |
| LIP148 | — | — | — | H | — | — | — | — | — | — | — | — | — | — | — | G | — | — | — |
| LIP147 | — | — | — | H | — | — | — | — | — | — | — | — | — | — | — | S | — | — | — |
| LIP146 | — | — | — | H | — | — | — | — | — | — | — | — | — | — | — | G | G | — | — |
| LIP145 | — | — | — | — | — | — | — | — | — | — | A | — | — | — | — | S | G | — | — |
| LIP144 | — | — | — | H | — | — | — | — | — | — | A | — | — | — | — | G | — | — | — |
| LIP143 | — | — | — | H | — | — | — | — | — | — | A | — | — | — | — | S | G | — | — |
| LIP142 | — | — | — | H | — | — | — | — | — | — | A | — | — | — | — | G | G | — | — |
| LIP135 | — | — | — | — | — | — | — | — | — | — | L | — | — | — | — | — | — | — | — |
| LIP134 | — | — | — | — | — | — | — | — | — | — | A | — | — | — | — | — | — | — | — |
| LIP131 | — | — | — | I | — | — | L | — | — | — | — | — | — | — | — | S | — | — | — |
| LIP130 | — | — | — | I | — | — | L | — | — | — | — | — | — | — | — | G | — | — | — |
| LIP126 | — | — | — | — | — | — | — | — | — | — | — | — | — | R | — | — | — | — | — |
| LIP124 | — | — | — | — | — | — | T | — | — | — | — | — | — | — | — | G | G | — | — |
| LIP123 | — | — | — | — | — | — | L | — | — | — | — | — | — | — | — | G | G | — | — |
| LIP120 | — | — | — | H | — | — | L | — | — | — | — | — | — | — | — | — | G | — | — |
| LIP119 | — | — | — | — | — | — | T | — | — | — | — | — | — | — | — | S | G | — | — |
| LIP118 | — | — | — | H | — | — | L | — | — | — | — | — | — | — | — | G | — | — | — |
| LIP117 | — | — | — | H | — | — | T | — | — | — | — | — | — | — | — | S | G | — | — |
| LIP116 | — | — | — | H | — | — | L | — | — | — | — | — | — | — | — | G | G | — | — |
| LIP115 | — | — | — | H | — | — | L | — | — | — | — | — | — | — | — | S | G | — | — |
| LIP114 | — | — | — | H | — | — | L | — | — | — | — | — | — | — | — | S | — | — | — |
| LIP113 | — | — | — | — | — | — | L | — | — | — | — | — | — | — | — | S | G | — | — |
| LIP111 | — | — | — | — | — | — | — | — | — | — | — | — | — | — | — | A | — | — | — |
| LIP110 | — | — | — | — | — | — | — | — | — | — | — | — | — | — | — | S | — | — | — |
| LIP109 | — | — | — | — | — | — | — | — | — | — | — | — | — | — | — | G | — | — | — |
| LIP108 | — | — | — | H | — | — | — | — | — | — | — | — | — | — | — | — | — | — | — |
| LIP102 | — | — | — | — | — | — | T | — | — | — | — | — | — | — | — | — | — | — | — |
| LIP101 | — | — | — | — | — | — | P | — | — | — | — | — | — | — | — | — | — | — | — |
| LIP100 | — | — | — | — | — | — | L | — | — | — | — | — | — | — | — | — | — | — | — |
| LIP099 | — | — | — | — | — | — | A | — | — | — | — | — | — | — | — | — | — | — | — |
| LIP096 | — | — | — | — | — | — | — | — | — | — | — | D | — | — | — | — | — | — | — |
| LIP095 | — | — | — | — | — | — | — | N | — | — | — | — | — | — | — | — | — | — | — |
| LIP094 | — | N | — | — | — | — | — | — | — | — | — | — | — | — | — | — | — | — | — |
| LIP090 | — | — | — | — | — | — | — | — | V | — | — | — | — | — | — | — | — | — | — |
| LIP089 | — | — | T | — | — | — | — | — | — | — | — | — | — | — | — | — | — | — | — |
| LIP062_ | — | — | — | — | — | — | T | — | — | — | A | — | — | — | — | — | — | — | — |
| 1909 | | | | | | | | | | | | | | | | | | | |
| LIP062_ | — | — | — | H | — | — | T | — | — | — | A | — | — | — | — | — | — | — | — |
| 1908 | | | | | | | | | | | | | | | | | | | |
| LIP062_ | — | — | — | — | — | — | P | — | — | — | A | — | — | — | — | S | — | — | — |
| 1907 | | | | | | | | | | | | | | | | | | | |
| LIP062_ | — | — | — | H | — | — | P | — | — | — | A | — | — | — | — | — | — | — | — |
| 1906 | | | | | | | | | | | | | | | | | | | |
| LIP062_ | — | — | — | I | — | — | — | — | — | — | A | — | — | — | — | G | G | — | — |
| 1905 | | | | | | | | | | | | | | | | | | | |
| LIP062_ | — | — | — | — | — | — | — | — | — | — | A | — | — | — | — | G | G | — | — |
| 1904 | | | | | | | | | | | | | | | | | | | |
| LIP062_ | — | — | — | H | — | — | P | — | — | — | — | — | — | — | — | — | G | — | — |
| 1903 | | | | | | | | | | | | | | | | | | | |
| LIP062_ | — | — | — | — | — | — | P | — | — | — | — | — | — | — | — | S | G | — | — |
| 1902 | | | | | | | | | | | | | | | | | | | |
| LIP062_ | — | — | — | — | — | — | T | — | — | — | — | — | — | — | — | S | — | — | — |
| 1901 | | | | | | | | | | | | | | | | | | | |
| LIP062_ | — | — | — | H | — | — | T | — | — | — | — | — | — | — | — | — | — | — | — |
| 1900 | | | | | | | | | | | | | | | | | | | |
| LIP062_ | — | — | — | — | — | — | P | — | — | — | — | — | — | — | — | S | — | — | — |
| 1899 | | | | | | | | | | | | | | | | | | | |
| LIP062_ | — | — | — | H | — | — | P | — | — | — | — | — | — | — | — | — | — | — | — |
| 1898 | | | | | | | | | | | | | | | | | | | |
| LIP062_ | — | — | — | I | — | — | — | — | — | — | — | — | — | — | — | G | — | — | — |
| 1897 | | | | | | | | | | | | | | | | | | | |
| LIP062_ | — | — | — | — | — | — | — | — | — | — | — | — | — | — | — | S | G | — | — |
| 1896 | | | | | | | | | | | | | | | | | | | |
| LIP062_ | — | — | — | I | — | — | — | — | — | — | — | — | — | — | — | G | G | — | — |
| 1895 | | | | | | | | | | | | | | | | | | | |
| LIP062_ | — | — | — | — | — | — | — | — | — | — | — | — | — | — | — | G | G | — | — |
| 1894 | | | | | | | | | | | | | | | | | | | |
| LIP062_ | — | — | — | I | — | — | T | — | — | — | — | — | — | — | — | — | G | — | — |
| 1893 | | | | | | | | | | | | | | | | | | | |
| LIP062_ | — | — | — | H | — | — | T | — | — | — | — | — | — | — | — | — | G | — | — |
| 1892 | | | | | | | | | | | | | | | | | | | |
| LIP062_ | — | — | — | I | — | — | T | — | — | — | — | — | — | — | — | S | — | — | — |
| 1891 | | | | | | | | | | | | | | | | | | | |
| LIP062_ | — | — | — | I | — | — | T | — | — | — | — | — | — | — | — | G | — | — | — |
| 1890 | | | | | | | | | | | | | | | | | | | |
| LIP062_ | — | — | — | H | — | — | T | — | — | — | — | — | — | — | — | S | — | — | — |
| 1889 | | | | | | | | | | | | | | | | | | | |
| LIP062_ | — | — | — | H | — | — | T | — | — | — | — | — | — | — | — | G | — | — | — |
| 1888 | | | | | | | | | | | | | | | | | | | |
| LIP062_ | — | — | — | I | — | — | L | — | — | — | — | — | — | — | — | — | G | — | — |
| 1887 | | | | | | | | | | | | | | | | | | | |
| LIP062_ | — | — | — | I | — | — | T | — | — | — | — | — | — | — | — | S | G | — | — |
| 1886 | | | | | | | | | | | | | | | | | | | |
| LIP062_ | — | — | — | I | — | — | L | — | — | — | — | — | — | — | — | S | G | — | — |
| 1885 | | | | | | | | | | | | | | | | | | | |
| LIP062_ | — | — | — | I | — | — | T | — | — | — | — | — | — | — | — | G | G | — | — |
| 1884 | | | | | | | | | | | | | | | | | | | |
| LIP062_ | — | — | — | I | — | — | L | — | — | — | — | — | — | — | — | G | G | — | — |
| 1883 | | | | | | | | | | | | | | | | | | | |
| LIP062_ | — | — | — | H | — | — | T | — | — | — | — | — | — | — | — | G | G | — | — |
| 1882 | | | | | | | | | | | | | | | | | | | |
| LIP062_ | — | — | — | — | — | — | — | — | — | — | — | — | — | — | I | — | — | — | — |
| 1881 | | | | | | | | | | | | | | | | | | | |
| LIP062_ | — | — | — | — | — | — | — | — | — | — | — | — | — | — | L | — | — | — | — |
| 1880 | | | | | | | | | | | | | | | | | | | |
| LIP062_ | — | — | — | — | — | — | — | — | — | — | — | — | — | P | — | — | — | — | — |
| 1879 | | | | | | | | | | | | | | | | | | | |
| LIP062_ | — | — | — | — | — | — | — | — | — | — | — | — | — | G | — | — | — | — | — |
| 1878 | | | | | | | | | | | | | | | | | | | |
| LIP062_ | — | — | — | — | — | — | — | — | — | — | — | — | — | S | — | — | — | — | — |
| 1877 | | | | | | | | | | | | | | | | | | | |
| LIP062_ | — | — | — | — | — | — | — | — | — | — | — | — | — | K | — | — | — | — | — |
| 1876 | | | | | | | | | | | | | | | | | | | |
| LIP062_ | — | — | — | I | N | — | V | — | — | — | — | — | — | — | — | — | — | — | — |
| 1875 | | | | | | | | | | | | | | | | | | | |
| LIP062_ | — | — | — | R | S | — | V | — | — | — | — | — | — | — | — | — | — | — | — |
| 1874 | | | | | | | | | | | | | | | | | | | |
| LIP062_ | — | — | — | — | — | — | — | — | — | — | — | — | — | — | — | — | L | — | — |
| 1873 | | | | | | | | | | | | | | | | | | | |
| LIP062_ | — | — | — | — | — | — | — | — | — | — | — | — | — | — | — | — | A | — | — |
| 1872 | | | | | | | | | | | | | | | | | | | |
| LIP062_ | — | — | — | — | — | — | — | — | — | — | — | — | — | — | — | — | N | — | — |
| 1871 | | | | | | | | | | | | | | | | | | | |
| LIP062_ | — | — | — | — | — | — | — | — | — | — | — | — | — | — | — | — | K | — | — |
| 1870 | | | | | | | | | | | | | | | | | | | |
| LIP062_ | — | — | — | — | — | — | — | — | — | — | — | — | — | — | — | — | S | — | — |
| 1869 | | | | | | | | | | | | | | | | | | | |
| LIP062_ | — | — | — | — | — | — | — | — | — | — | — | — | — | — | — | — | G | — | — |
| 1868 | | | | | | | | | | | | | | | | | | | |
| LIP062_ | — | — | — | — | — | — | — | — | — | — | — | — | — | — | — | L | — | — | — |
| 1867 | | | | | | | | | | | | | | | | | | | |
| LIP062_ | — | — | — | — | — | — | — | — | — | — | — | — | — | — | — | N | — | — | — |
| 1866 | | | | | | | | | | | | | | | | | | | |
| LIP062_ | — | — | — | — | — | — | — | — | — | — | — | — | — | — | — | K | — | — | — |
| 1865 | | | | | | | | | | | | | | | | | | | |
| LIP062_ | — | — | — | N | — | — | — | — | — | — | — | — | — | — | — | — | — | — | — |
| 1864 | | | | | | | | | | | | | | | | | | | |
| LIP062_ | — | — | — | D | — | — | — | — | — | — | — | — | — | — | — | — | — | — | — |
| 1863 | | | | | | | | | | | | | | | | | | | |
| LIP062_ | — | — | — | I | — | — | — | — | — | — | — | — | — | — | — | — | — | — | — |
| 1862 | | | | | | | | | | | | | | | | | | | |
| LIP062_ | A | — | — | — | — | — | — | — | — | — | — | — | — | — | — | — | — | — | — |
| 1861 | | | | | | | | | | | | | | | | | | | |
| LIP062_ | — | — | — | — | — | — | — | — | — | — | — | — | — | — | — | — | — | A | E |
| 1860 | | | | | | | | | | | | | | | | | | | |
| LIP062_ | — | — | — | — | — | — | — | — | — | — | — | — | — | — | — | — | — | A | — |
| 1859 | | | | | | | | | | | | | | | | | | | |
| LIP062_ | — | — | — | — | — | — | — | — | — | — | — | — | — | — | — | — | — | — | E |
| 1858 | | | | | | | | | | | | | | | | | | | |
| LIP062_ | — | — | — | — | S | — | — | — | — | — | — | — | — | — | — | — | — | — | — |
| 1857 | | | | | | | | | | | | | | | | | | | |
| LIP062_ | — | — | — | — | L | — | — | — | — | — | — | — | — | — | — | — | — | — | — |
| 1856 | | | | | | | | | | | | | | | | | | | |
| LIP062_ | — | — | — | — | Y | — | — | — | — | — | — | — | — | — | — | — | — | — | — |
| 1855 | | | | | | | | | | | | | | | | | | | |
| LIP062_ | — | — | — | — | — | — | — | — | — | — | — | — | E | — | — | — | — | — | — |
| 1854 | | | | | | | | | | | | | | | | | | | |
| LIP062_ | — | — | — | — | — | — | — | — | — | — | — | — | Q | — | — | — | — | — | — |
| 1853 | | | | | | | | | | | | | | | | | | | |
| LIP062_ | — | — | — | — | — | — | — | — | — | — | — | — | T | — | — | — | — | — | — |
| 1852 | | | | | | | | | | | | | | | | | | | |
| LIP062_ | — | — | — | — | — | — | — | — | — | — | — | — | H | — | — | — | — | — | — |
| 1851 | | | | | | | | | | | | | | | | | | | |
| LIP062_ | — | — | — | — | — | — | — | — | — | — | — | — | D | — | — | — | — | — | — |
| 1850 | | | | | | | | | | | | | | | | | | | |
| LIP062_ | — | — | — | — | — | — | — | — | — | — | — | — | V | — | — | — | — | — | — |
| 1849 | | | | | | | | | | | | | | | | | | | |
| LIP062_ | — | — | — | — | — | — | — | — | — | — | — | — | R | — | — | — | — | — | — |
| 1848 | | | | | | | | | | | | | | | | | | | |
| LIP062_ | — | — | — | — | — | — | — | — | — | — | — | — | N | — | — | — | — | — | — |
| 1847 | | | | | | | | | | | | | | | | | | | |
| LIP062_ | — | — | — | — | — | — | — | — | — | — | — | — | G | — | — | — | — | — | — |
| 1846 | | | | | | | | | | | | | | | | | | | |
| LIP062_ | — | — | — | — | — | — | — | — | — | — | — | — | A | — | — | — | — | — | — |
| 1845 | | | | | | | | | | | | | | | | | | | |
| LIP062_ | — | — | — | — | — | — | — | — | — | — | — | — | S | — | — | — | — | — | — |
| 1844 | | | | | | | | | | | | | | | | | | | |
| LIP062_ | — | — | — | — | — | — | — | — | — | M | — | — | — | — | — | — | — | — | — |
| 1843 | | | | | | | | | | | | | | | | | | | |
| LIP062_ | — | — | — | — | — | — | — | — | — | G | — | — | — | — | — | — | — | — | — |
| 1842 | | | | | | | | | | | | | | | | | | | |
| LIP062_ | — | — | — | — | — | — | — | — | — | R | — | — | — | — | — | — | — | — | — |
| 1841 | | | | | | | | | | | | | | | | | | | |
| LIP062_ | — | — | — | — | — | — | — | — | — | F | — | — | — | — | — | — | — | — | — |
| 1840 | | | | | | | | | | | | | | | | | | | |
| LIP062_ | — | — | — | — | — | — | — | — | — | E | — | — | — | — | — | — | — | — | — |
| 1839 | | | | | | | | | | | | | | | | | | | |
| LIP062_ | — | — | — | — | — | — | — | — | — | W | — | — | — | — | — | — | — | — | — |
| 1838 | | | | | | | | | | | | | | | | | | | |
| LIP062_ | — | — | — | — | — | — | — | — | — | L | — | — | — | — | — | — | — | — | — |
| 1837 | | | | | | | | | | | | | | | | | | | |
| LIP062_ | — | — | — | — | — | — | — | — | — | Y | — | — | — | — | — | — | — | — | — |
| 1836 | | | | | | | | | | | | | | | | | | | |
| LIP062_ | — | — | — | — | — | — | — | — | — | S | — | — | — | — | — | — | — | — | — |
| 1835 | | | | | | | | | | | | | | | | | | | |
| LIP062_ | — | — | — | — | — | — | — | — | — | C | — | — | — | — | — | — | — | — | — |
| 1834 | | | | | | | | | | | | | | | | | | | |
| LIP062_ | — | — | — | — | — | — | — | — | — | A | — | — | — | — | — | — | — | — | — |
| 1833 | | | | | | | | | | | | | | | | | | | |
| LIP062_ | — | — | — | — | — | — | — | — | — | V | — | — | — | — | — | — | — | — | — |
| 1832 | | | | | | | | | | | | | | | | | | | |
| LIP062_ | — | — | — | — | — | — | — | — | — | N | — | — | — | — | — | — | — | — | — |
| 1831 | | | | | | | | | | | | | | | | | | | |
| LIP062_ | — | — | — | — | — | — | M | — | — | — | — | — | — | — | — | — | — | — | — |
| 1830 | | | | | | | | | | | | | | | | | | | |
| LIP062_ | — | — | — | — | — | — | S | — | — | — | — | — | — | — | — | — | — | — | — |
| 1829 | | | | | | | | | | | | | | | | | | | |
| LIP062_ | — | — | — | — | — | — | C | — | — | — | — | — | — | — | — | — | — | — | — |
| 1828 | | | | | | | | | | | | | | | | | | | |
| LIP062_ | — | N | — | — | — | — | — | N | — | — | — | — | — | — | — | — | — | — | — |
| 1827 | | | | | | | | | | | | | | | | | | | |
| LIP062_ | — | — | — | — | — | — | — | — | I | — | — | — | — | — | — | — | — | — | — |
| 1826 | | | | | | | | | | | | | | | | | | | |
| LIP062_ | — | — | — | N | — | — | V | — | — | — | A | — | — | — | A | G | — | — | — |
| 1825 | | | | | | | | | | | | | | | | | | | |
| LIP062_ | — | — | — | T | — | — | V | — | — | — | A | — | — | — | — | G | — | — | — |
| 1824 | | | | | | | | | | | | | | | | | | | |
| LIP062_ | — | — | — | N | — | — | V | — | — | — | A | — | — | — | S | S | — | — | — |
| 1823 | | | | | | | | | | | | | | | | | | | |
| LIP062_ | — | — | — | H | — | — | T | — | — | — | A | — | — | — | S | S | — | — | — |
| 1822 | | | | | | | | | | | | | | | | | | | |
| LIP062_ | — | — | — | — | — | — | — | — | — | — | A | — | — | — | A | T | — | — | — |
| 1820 | | | | | | | | | | | | | | | | | | | |
| LIP062_ | — | — | — | Y | — | — | — | — | — | — | A | — | — | — | — | T | — | — | — |
| 1818 | | | | | | | | | | | | | | | | | | | |
| LIP062_ | — | — | — | — | — | — | — | — | — | — | A | — | — | — | G | T | — | — | — |
| 1817 | | | | | | | | | | | | | | | | | | | |
| LIP062_ | — | — | — | — | — | — | — | — | — | — | A | — | — | — | N | A | — | — | — |
| 1816 | | | | | | | | | | | | | | | | | | | |
| LIP062_ | — | — | — | T | — | — | A | — | — | — | A | — | — | — | — | T | — | — | — |
| 1814 | | | | | | | | | | | | | | | | | | | |
| LIP062_ | — | — | — | N | — | — | — | — | — | — | A | — | — | — | — | A | — | — | — |
| 1812 | | | | | | | | | | | | | | | | | | | |
| LIP062_ | — | — | — | T | — | — | — | — | — | — | A | — | — | — | N | T | — | — | — |
| 1810 | | | | | | | | | | | | | | | | | | | |
| LIP062_ | — | — | — | — | — | — | — | — | — | — | A | — | — | — | D | A | — | — | — |
| 1807 | | | | | | | | | | | | | | | | | | | |
| LIP062_ | — | — | — | H | — | — | V | — | — | — | A | — | — | — | — | A | — | — | — |
| 1805 | | | | | | | | | | | | | | | | | | | |
| LIP062_ | — | — | — | H | — | — | — | — | — | — | A | — | — | — | A | T | — | — | — |
| 1804 | | | | | | | | | | | | | | | | | | | |
| LIP062_ | — | — | — | N | — | — | V | — | — | — | A | — | — | — | S | A | — | — | — |
| 1803 | | | | | | | | | | | | | | | | | | | |
| LIP062_ | — | — | — | — | — | — | — | — | — | — | A | — | — | — | — | G | — | — | — |
| 1801 | | | | | | | | | | | | | | | | | | | |
| LIP062_ | — | — | — | — | — | — | — | — | — | — | A | — | — | — | N | T | — | — | — |
| 1799 | | | | | | | | | | | | | | | | | | | |
| LIP062_ | — | — | — | Y | — | — | V | — | — | — | A | — | — | — | N | T | — | — | — |
| 1798 | | | | | | | | | | | | | | | | | | | |
| LIP062_ | — | — | — | H | — | — | T | — | — | — | A | — | — | — | — | A | — | — | — |
| 1797 | | | | | | | | | | | | | | | | | | | |
| LIP062_ | — | — | — | H | — | — | — | — | — | — | A | — | — | — | A | S | — | — | — |
| 1796 | | | | | | | | | | | | | | | | | | | |
| LIP062_ | — | — | — | N | — | — | V | — | — | — | A | — | — | — | N | T | — | — | — |
| 1795 | | | | | | | | | | | | | | | | | | | |
| LIP062_ | — | — | — | — | — | — | — | — | — | — | A | — | — | — | — | T | — | — | — |
| 1793 | | | | | | | | | | | | | | | | | | | |
| LIP062_ | — | — | — | Y | — | — | V | — | — | — | A | — | — | — | S | T | — | — | — |
| 1792 | | | | | | | | | | | | | | | | | | | |
| LIP062_ | — | — | — | — | — | — | — | — | — | — | A | — | — | — | S | S | — | — | — |
| 1790 | | | | | | | | | | | | | | | | | | | |
| LIP062_ | — | — | — | N | — | — | L | — | — | — | A | — | — | — | S | G | — | — | — |
| 1788 | | | | | | | | | | | | | | | | | | | |
| LIP062_ | — | — | — | N | — | — | — | — | — | L | — | — | — | — | N | T | — | — | — |
| 1782 | | | | | | | | | | | | | | | | | | | |
| LIP062_ | — | — | — | H | — | — | A | — | — | L | — | — | — | — | A | T | — | — | — |
| 1781 | | | | | | | | | | | | | | | | | | | |
| LIP062_ | — | — | — | H | — | — | — | — | — | L | — | — | — | — | — | G | — | — | — |
| 1780 | | | | | | | | | | | | | | | | | | | |
| LIP062_ | — | — | — | N | — | — | V | — | — | L | — | — | — | — | D | T | — | — | — |
| 1779 | | | | | | | | | | | | | | | | | | | |
| LIP062_ | — | — | — | — | — | — | — | — | — | L | — | — | — | — | A | T | — | — | — |
| 1778 | | | | | | | | | | | | | | | | | | | |
| LIP062_ | — | — | — | H | — | — | V | — | — | L | — | — | — | — | — | A | — | — | — |
| 1776 | | | | | | | | | | | | | | | | | | | |
| LIP062_ | — | — | — | T | — | — | V | — | — | L | — | — | — | — | S | A | — | — | — |
| 1775 | | | | | | | | | | | | | | | | | | | |
| LIP062_ | — | — | — | — | — | — | — | — | — | L | — | — | — | — | D | A | — | — | — |
| 1774 | | | | | | | | | | | | | | | | | | | |
| LIP062_ | — | — | — | N | — | — | V | — | — | L | — | — | — | — | A | A | — | — | — |
| 1773 | | | | | | | | | | | | | | | | | | | |
| LIP062_ | — | — | — | — | — | — | — | — | — | L | — | — | — | — | N | T | — | — | — |
| 1770 | | | | | | | | | | | | | | | | | | | |
| LIP062_ | — | — | — | — | — | — | — | — | — | L | — | — | — | — | D | T | — | — | — |
| 1768 | | | | | | | | | | | | | | | | | | | |
| LIP062_ | — | — | — | — | — | — | — | — | — | L | — | — | — | — | S | T | — | — | — |
| 1767_ | | | | | | | | | | | | | | | | | | | |
| LIP062_ | — | — | — | — | — | — | — | — | — | L | — | — | — | — | N | A | — | — | — |
| 1766 | | | | | | | | | | | | | | | | | | | |
| LIP062_ | — | — | — | H | — | — | — | — | — | — | — | — | — | — | A | A | — | — | — |
| 1704 | | | | | | | | | | | | | | | | | | | |
| LIP062_ | — | — | — | H | — | — | T | — | — | — | — | — | — | — | A | A | — | — | — |
| 1703 | | | | | | | | | | | | | | | | | | | |
| LIP062_ | — | — | — | T | — | — | V | — | — | — | — | — | — | — | G | T | — | — | — |
| 1701 | | | | | | | | | | | | | | | | | | | |
| LIP062_ | — | — | — | — | — | — | — | — | — | — | — | — | — | — | S | T | — | — | — |
| 1700 | | | | | | | | | | | | | | | | | | | |
| LIP062_ | — | — | — | — | — | — | — | — | — | — | — | — | — | — | A | T | — | — | — |
| 1696 | | | | | | | | | | | | | | | | | | | |
| LIP062_ | — | — | — | N | — | — | V | — | — | — | — | — | — | — | A | T | — | — | — |
| 1695 | | | | | | | | | | | | | | | | | | | |
| LIP062_ | — | — | — | — | — | — | — | — | — | — | — | — | — | — | — | G | — | — | — |
| 1694 | | | | | | | | | | | | | | | | | | | |
| LIP062_ | — | — | — | — | — | — | — | — | — | — | — | — | — | — | A | T | — | — | — |
| 1692 | | | | | | | | | | | | | | | | | | | |
| LIP062_ | — | — | — | N | — | — | V | — | — | — | — | — | — | — | S | S | — | — | — |
| 1691 | | | | | | | | | | | | | | | | | | | |
| LIP062_ | — | — | — | H | — | — | V | — | — | — | — | — | — | — | A | S | — | — | — |
| 1686 | | | | | | | | | | | | | | | | | | | |
| LIP062_ | — | — | — | N | — | — | V | — | — | — | — | — | — | — | N | A | — | — | — |
| 1685 | | | | | | | | | | | | | | | | | | | |
| LIP062_ | — | — | — | — | — | — | — | — | — | — | — | — | — | — | N | T | — | — | — |
| 1684 | | | | | | | | | | | | | | | | | | | |
| LIP062_ | — | — | — | — | — | — | — | — | — | — | — | — | — | — | D | A | — | — | — |
| 1683 | | | | | | | | | | | | | | | | | | | |
| LIP062_ | — | — | — | T | — | — | — | — | — | — | — | — | — | — | N | T | — | — | — |
| 1681 | | | | | | | | | | | | | | | | | | | |
| LIP062_ | — | — | — | N | — | — | A | — | — | — | — | — | — | — | — | T | — | — | — |
| 1680 | | | | | | | | | | | | | | | | | | | |
| LIP062_ | — | — | — | N | — | — | — | — | — | — | — | — | — | — | — | A | — | — | — |
| 1678 | | | | | | | | | | | | | | | | | | | |
| LIP062_ | — | — | — | — | — | — | — | — | — | — | — | — | — | — | G | T | — | — | — |
| 1677 | | | | | | | | | | | | | | | | | | | |
| LIP062_ | — | — | — | Y | — | — | — | — | — | — | — | — | — | — | G | T | — | — | — |
| 1676 | | | | | | | | | | | | | | | | | | | |
| LIP062_ | — | — | — | — | — | — | — | — | — | — | — | — | — | — | G | T | — | — | — |
| 1674 | | | | | | | | | | | | | | | | | | | |
| LIP062_ | — | — | — | N | — | — | — | — | — | — | — | — | — | — | — | T | — | — | — |
| 1670 | | | | | | | | | | | | | | | | | | | |
| LIP062_ | — | — | — | — | — | — | — | — | — | — | — | — | — | — | S | G | — | — | — |
| 1669 | | | | | | | | | | | | | | | | | | | |
| LIP062_ | — | — | — | N | — | — | — | — | — | — | — | — | — | — | — | G | — | — | — |
| 1668 | | | | | | | | | | | | | | | | | | | |
| LIP062_ | — | — | — | — | — | — | A | — | — | — | — | — | — | — | — | G | — | — | — |
| 1667 | | | | | | | | | | | | | | | | | | | |
| LIP062_ | — | — | — | — | — | — | — | — | — | — | — | — | — | — | D | T | — | — | — |
| 1665 | | | | | | | | | | | | | | | | | | | |
| LIP062_ | — | — | — | N | — | — | — | — | — | L | — | — | — | — | A | T | — | — | — |
| 1664 | | | | | | | | | | | | | | | | | | | |
| LIP062_ | — | — | — | — | — | — | F | — | — | — | — | — | — | — | — | — | — | — | — |
| 0450 | | | | | | | | | | | | | | | | | | | |
| LIP062_ | — | — | — | — | — | — | Y | — | — | — | — | — | — | — | — | — | — | — | — |
| 0449 | | | | | | | | | | | | | | | | | | | |
| LIP062_ | — | — | — | — | — | — | — | — | — | — | — | — | — | — | — | — | — | — | — |
| 0391 | | | | | | | | | | | | | | | | | | | |

The term “promoter” as used herein refers to a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription may also be characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O′Reilly et al., J. Biol. Chem. 267:19938 (1992)), AP2 (Ye et al., J. Biol. Chem. 269:25728 (1994)), SP1, CAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987;), and Lemaigre and Rousseau, Biochem. J. 303:1 (1994)).

In the context of the present invention, the promoter sequence used in the first and optionally the second expression cassette to express the recombinant protein comprises a nucleic acid sequence selected from the group consisting of SEQ ID Nos. 1-8 (pSD001 (SEQ ID No. 1), pSD002 (SEQ ID No. 2), pSD003 (SEQ ID No. 3), pSD004 (SEQ ID No. 4), pSD005 (SEQ ID No. 5), pSD007 (SEQ ID No. 6), pSD008 (SEQ ID No. 7) and AOX (SEQ ID No. 8).

In one embodiment, the promoter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% or more sequence identity to any one of SEQ ID Nos. 1-7 or a fragment thereof, and drives protein expression in a yeast cell in the absence of methanol. In some embodiments, the sequence identity is over a region of at least 50, 75, 100, 150, 200, 250, 300, 350, 400, 450, 500, 550, 600, 650, 700, 750, 800, 850, 900, 950, 1000, 2050, 1100, 1150, or more residues, or the full length of the nucleic acid. In some embodiments, the fragment of any one of SEQ ID Nos. 1-7 is over a region of at least 50, 75, 100, 150, 200, 250, 300, 350, 400, 450, 500, 550, 600, 650, 700, 750, 800, 850, 900, 950, 1000, 2050, 1100, 1150, or more residues, or the full length of the nucleic acid and drives protein expression in a yeast cell in the absence of methanol.

In one embodiment, the promoter comprises a functional fragment of the nucleic acid sequence according to any one of SEQ ID Nos. 1-7. The functional fragment comprises at least 50, 75, 100, 150, 200, 250, 300, 350, 400, 450, 500, 550, 600, 650, 700, 750, 800, 850, 900, 950, 1000, 2050, 1100 or 1150 contiguous nucleotides of any of the sequences according to any one of SEQ ID Nos. 1-7. The term “functional” is intended to mean that the promoter is useful in driving protein expression independently of methanol, wherein the promoter drives protein expression in a methanol-free medium.

In some embodiments, the promoter is a promoter that is useful in driving protein expression independently of methanol, wherein the promoter drives protein expression in a methanol-free medium. This means that the promoter is active in the absence of methanol. The expression “promoter is active in the absence of methanol” is used herein interchangeably with “promoter drives protein expression independently of methanol” and “promoter that allows an increase in protein expression in the absence of methanol”.

In one embodiment, the promoter sequence comprises the nucleic acid sequence according to SEQ ID No. 1. In one embodiment, the promoter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% or more sequence identity to SEQ ID No. 1 or a fragment thereof, and the promoter drives protein expression in a yeast cell in absence of methanol.

In one embodiment, the promoter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% or more sequence identity to SEQ ID No. 8 or a fragment thereof, and drives protein expression in a yeast cell in the presence of methanol. In some embodiments, the sequence identity is over a region of at least 50, 75, 100, 150, 200, 250, 300, 350, 400, 450, 500, 550, 600, 650, 700,750, 800, 850, 900, 950, 1000, 2050, 1100, 1150, or more residues, or the full length of the nucleic acid. In some embodiments, the fragment of SEQ ID No. 8 is over a region of at least 50, 75, 100, 150, 200, 250, 300, 350, 400, 450, 500, 550, 600, 650, 700, 750, 800, 850, 900, 950, 1000, 2050, 1100, 1150, or more residues, or the full length of the nucleic acid and drives protein expression in a yeast cell in the presence of methanol.

The promoter may be operably linked to the nucleic acid molecule encoding the leader peptide and the nucleic acid molecule encoding the recombinant protein so that the promoter is capable of effecting expression of the leader peptide and the recombinant protein.

In one embodiment the nucleic acid sequences operably linked to each other are immediately linked, i.e. without further elements or nucleic acid sequences between the promoter and the nucleic acid sequence encoding the protein and the leader peptide.

In one embodiment, the first expression cassette comprises a nucleic acid sequence encoding the first auxotrophic marker which is HIS4 (histidinol dehydrogenase). In one embodiment, the nucleic acid sequence encoding HIS4 is from Saccharomyces cerevisiae. In one embodiment, the nucleic acid sequence encoding HIS4 is the nucleic acid sequence according to SEQ ID No. 25 or a functional variant thereof which has at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% or more sequence identity to the nucleic acid sequence according to SEQ ID No. 25. In one embodiment, the nucleic acid sequence encodes a polypeptide having at least 80%, 81%, 82%, 83%, 84%, 85%, 86%, 87%, 88%, 89%, 90%, 91%, 92%, 93%, 94%, 95%, 96%, 97%, 98% or 99% or more sequence identity to the amino acid sequence according to SEQ ID No. 27. The functional variant of the nucleic acid sequence according to SEQ ID No. 25 encodes a protein with histidinol dehydrogenase activity. Hence, the functional variant is able to complement the his4-deficient phenotype of the yeast cell and allows the yeast cells to grow on a medium which does not comprise histidine.

In one embodiment, the second expression cassette comprises a nucleic acid sequence encoding the second auxotrophic marker which is URA3 (orotidine 5′ phosphate decarboxylase). In one embodiment, the nucleic acid sequence encoding URA3 is from Saccharomyces cerevisiae. In one embodiment, the nucleic acid sequence encoding URA3 is the nucleic acid sequence according to SEQ ID No. 26 or a functional variant thereof which has at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% or more sequence identity to the nucleic acid sequence according to SEQ ID No. 26. In one embodiment, the nucleic acid sequence encodes a polypeptide having at least 80%, 81%, 82%, 83%, 84%, 85%, 86%, 87%, 88%, 89%, 90%, 91%, 92%, 93%, 94%, 95%, 96%, 97%, 98% or 99% or more sequence identity to the amino acid sequence according to SEQ ID No. 28. The functional variant of the nucleic acid sequence according to SEQ ID No. 26 encodes a protein with orotidine 5′ phosphate decarboxylase activity. Hence, the functional variant is able to complement the ura3-deficient phenotype of the yeast cell and allows the yeast cells to grow on a medium which does not comprise uracil or uridine.

The first and optionally second expression cassette is stably integrated into the genome of the yeast cell, preferably the Komagataella phaffii cell. This means that the first and optionally second expression cassette is replicated together with the genome of the yeast cell and is not lost during cell division.

The term “vector” refers to DNA sequences that are required for the transcription of cloned recombinant nucleotide sequences, i.e. of recombinant genes and the translation of their mRNA in a suitable host organism. Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together.

The term “vector” as used herein includes autonomously replicating nucleotide sequences as well as genome integrating nucleotide sequences. Vectors include, but are not limited to, plasmids, minicircles, yeast integrative plasmids, episomal plasmids, centromere plasmids, artificial chromosomes and viral genomes. Available commercial vectors are known to those of skill in the art. Commercial vectors are available from European Molecular Biology Laboratory and Atum, for example.

In a preferred embodiment, the expression vector is a plasmid suitable for integration into the genome of the host cell, in a single copy or in multiple copies per cell. The nucleic acid sequence encoding the promoter, the recombinant protein and the auxotrophic marker may also be provided on an autonomously replicating plasmid in a single copy or in multiple copies per cell. The preferred plasmid is a eukaryotic expression vector, preferably a yeast expression vector. The expression vector may be any vector which is capable of replicating in or integrating into the genome of the host organisms. Preferably, the vector is functional in yeast cells such as Komagataella phaffii cells.

The vector can be produced by any method known in the art. For example, procedures to ligate the nucleic acid sequences encoding a protein and to insert the ligated sequences into a suitable vector are known and described for example in Green and Sambrook (2012) Molecular Cloning, 4th edition, Cold Spring Harbor Laboratory Press.

In one embodiment, a method for producing a recombinant protein in the recombinant yeast cell of the present invention is provided. According to the method, the yeast cell is cultured under suitable conditions, before the protein is obtained. Suitable conditions are those that permit expression and secretion of the protein and are well-known to the person skilled in the art. They include cultivation in the batch mode, the fed-batch mode and the continuous mode.

The host cell may be cultured on an industrial scale which may employ culture medium volumina of at least 10 litres, preferably of at least 50 litres and most preferably of at least 100 litres.

The host cell may be cultured under growth conditions to obtain a cell density of at least 1 g/L cell dry weight, more preferably at least 10 g/L cell dry weight, preferably at least 20 g/L cell dry weight.

The nutrient broth used for cell culture may comprise at least one carbon source. In some embodiments, the at least one carbon source is selected from a group consisting of dextrose, maltose, glucose, dextrin, glycerol, sorbitol, mannitol, lactic acid, acetate, xylose, or other partially hydrolyzed starches, and any mixtures thereof. In some embodiments, the concentrations of the at least one carbon source varies from 0.0 g/1, 0.5g/L, 1g/L, 2 g/L, 4 g/L, 6 g/L, 8 g/L, 10 g/L, 11 g/L, 12 g/L, 13 g/L, 14 g/L, 15 g/L, 16 g/L, 18 g/L, 20 g/L, 22 g/L, 24 g/L, 26 g/L, 28g/L, 30 g/L, 35 g/L, 40 g/L, 45 g/L, 50 g/L, 55g/L, or 60 g/L any concentration within a range defined by any two aforementioned values. In some embodiments, the method further comprises addition of the at least one carbon source by pulse or continuous feeding.

The protein produced by the host cell may be obtained by any known process for isolating and purifying proteins. Such processes include, but are not limited to, salting out and solvent precipitation, ultrafiltration, gel electrophoresis, ion-exchange chromatography, affinity chromatography, reverse phase high performance liquid chromatography, hydrophobic interaction chromatography, mixed mode chromatography, hydroxyapatite chromatography and isoelectric focusing.

The titer of the recombinant protein in the supernatant of the recombinant yeast cells is from 0.2 to 15.5 g/l or from 0.5 to 15.5 g/l or from 1.0 to 15.5 g/l or from 1.5 to 15.5 g/l or from 2.0 to 15.5 g/l or from 2.5 to 15.5 g/l or from 3.0 to 15.5 g/l or from 3.5 to 15.5 g/l or from 4.0 to 15.5 g/l or from 4.5 to 15.5 g/l or from 5.0 to 15.5 g/l or from 5.3 to 15.5 g/1. The titer of the recombinant protein in the supernatant of the recombinant yeast cells is from 0.8 g/l to 15.5 g/l or from 0.8 g/l to 15.0 g/l or from 0.8 g/l to 14.5 g/l or from 0.8 g/l to 14.0 g/l or from 0.8 g/l to 13.5 g/l or from 0.8 g/l to 13.0 g/l or from 0.8 g/l to 12.5 g/l or from 0.8 g/l to 12.0 g/l or from 0.8 g/l to 11.5 g/l or from 0.8 g/l to 11.0 g/l or from 0.8 g/l to 10.5 g/l or from 0.8 g/l to 10.0 g/l or from 0.8 g/l to 9.5 g/l or from 0.8 g/l to 9.0 g/l or from 0.8 g/l to 8.5 g/l or from 0.8 g/l to 8.0 g/l or from 0.8 g/l to 7.5 g/l or from 0.8 g/l to 7.0 g/l or from 0.8 g/l to 6.5 g/l or from 0.8 g/l to 6.0 g/l. The titer of the recombinant protein in the supernatant of the recombinant yeast cells is from 3.8 to 5.4 g/l or from 0.9 to 8.8 g/l or from 1.7 to 2.5 g/l or from 1.1 to 11.8 g/l or from 2.8 to 9.0 g/l or from 3.5 to 13.7 g/l or from 1.8 to 2.7 g/l or from 5.3 to 5.7 g/l or from 3.2 to 6.1 g/l or from 0.2 to 3.5 g/l or from 4.4 to 15.5 g/l or from 0.9 to 12.8 g/l or from 0.8 to 5.2 g/l or from 0.6 to 11.1 g/l or from 1.6 to 2.5 g/l or from 0.8 to 1.9 g/l or from 3.1 to 6.9 g/l or from 2.9 to 4.9 g/l.

In one embodiment, the present invention provides a recombinant Komagataella phaffii cell which is deficient for a first auxotrophic marker and a second auxotrophic marker, wherein the recombinant yeast cell is transformed with:
(a) a first expression cassette which comprises a nucleic acid sequence encoding a recombinant protein operatively linked to a promoter according to SEQ ID No. 1, and a nucleic acid sequence encoding said first auxotrophic marker; and
(b) a second expression cassette which comprises a nucleic acid sequence encoding said recombinant protein operatively linked to a promoter according to SEQ ID No. 1, and a nucleic acid sequence encoding said second auxotrophic marker.

In one embodiment, the present invention provides a recombinant Komagataella phaffii cell which is deficient for a first auxotrophic marker and a second auxotrophic marker, wherein the recombinant yeast cell is transformed with:
(a) a first expression cassette which comprises a nucleic acid sequence encoding a recombinant lipase operatively linked to a promoter according to SEQ ID No. 1, and a nucleic acid sequence encoding said first auxotrophic marker; and
(b) a second expression cassette which comprises a nucleic acid sequence encoding said recombinant lipase operatively linked to a promoter according to SEQ ID No. 1, and a nucleic acid sequence encoding said second auxotrophic marker.

In one embodiment, the present invention provides a recombinant Komagataella phaffii cell which is deficient for a first auxotrophic marker and a second auxotrophic marker, wherein the recombinant yeast cell is transformed with:
(a) a first expression cassette which comprises a nucleic acid sequence encoding a recombinant protein fused to a nucleic acid sequence encoding a leader peptide and operatively linked to a promoter according to SEQ ID No. 1, and a nucleic acid sequence encoding said first auxotrophic marker; and
(b) a second expression cassette which comprises a nucleic acid sequence encoding said recombinant protein fused to a nucleic acid sequence encoding a leader peptide and operatively linked to a promoter according to SEQ ID No. 1, and a nucleic acid sequence encoding said second auxotrophic marker.

In one embodiment, the present invention provides a recombinant Komagataella phaffii cell which is deficient for a first auxotrophic marker and a second auxotrophic marker, wherein the recombinant yeast cell is transformed with:
(a) a first expression cassette which comprises a nucleic acid sequence encoding a recombinant protein fused to a nucleic acid sequence encoding the leader peptide according to SEQ ID No. 9 and operatively linked to a promoter according to SEQ ID No. 1, and a nucleic acid sequence encoding said first auxotrophic marker; and
(b) a second expression cassette which comprises a nucleic acid sequence encoding said recombinant protein fused to a nucleic acid sequence encoding the leader peptide according to SEQ ID No. 9 and operatively linked to a promoter according to SEQ ID No. 1, and a nucleic acid sequence encoding said second auxotrophic marker.

In one embodiment, the present invention provides a recombinant Komagataella phaffii cell which is deficient for his4 and ura3, wherein said cell is transformed with:
(a) a first expression cassette which comprises a nucleic acid sequence encoding a recombinant protein operatively linked to a promoter that is at least 80% identical to the sequence according to any one of SEQ ID Nos. 1-8, and a nucleic acid sequence encoding HIS4; and
(b) a second expression cassette which comprises a nucleic acid sequence encoding said recombinant protein operatively linked to a promoter that is at least 80% identical to the sequence according to any one of SEQ ID Nos. 1-8, and a nucleic acid sequence encoding URA3.

In one embodiment, the present invention provides a recombinant Komagataella phaffii cell which is deficient for his4 and ura3, wherein said cell is transformed with:
(a) a first expression cassette which comprises a nucleic acid sequence encoding a recombinant protein operatively linked to a promoter according to SEQ ID No. 1, and a nucleic acid sequence encoding HIS4; and
(b) a second expression cassette which comprises a nucleic acid sequence encoding a recombinant protein operatively linked to a promoter according to SEQ ID No. 1, and a nucleic acid sequence encoding URA3.

In one embodiment, the present invention provides a recombinant Komagataella phaffii cell which is deficient for his4 and ura3, wherein said cell is transformed with:
(a) a first expression cassette which comprises a nucleic acid sequence encoding a lipase operatively linked to a promoter according to SEQ ID No. 1, and a nucleic acid sequence encoding HIS4; and
(b) a second expression cassette which comprises a nucleic acid sequence encoding a lipase operatively linked to a promoter according to SEQ ID No. 1, and a nucleic acid sequence encoding URA3.

In one embodiment, the present invention provides a recombinant Komagataella phaffii cell which is deficient for his4 and ura3, wherein said cell is transformed with:
(a) a first expression cassette which comprises a nucleic acid sequence encoding a recombinant protein fused to a nucleic acid sequence encoding a leader peptide and operatively linked to a promoter according to SEQ ID No. 1, and a nucleic acid sequence encoding HIS4; and
(b) a second expression cassette which comprises a nucleic acid sequence encoding a recombinant protein fused to a nucleic acid sequence encoding a leader peptide operatively linked to a promoter according to SEQ ID No. 1, and a nucleic acid sequence encoding URA3.

In one embodiment, the present invention provides a recombinant Komagataella phaffii cell which is deficient for his4 and ura3, wherein said cell is transformed with:
(a) a first expression cassette which comprises a nucleic acid sequence encoding a recombinant protein fused to a nucleic acid sequence encoding the leader peptide according to SEQ ID No. 9 and operatively linked to a promoter according to SEQ ID No. 1, and a nucleic acid sequence encoding HIS4; and
(b) a second expression cassette which comprises a nucleic acid sequence encoding a recombinant protein fused to a nucleic acid sequence encoding the leader peptide according to SEQ ID No. 9 and operatively linked to a promoter according to SEQ ID No. 1, and a nucleic acid sequence encoding URA3.

In one embodiment, the present invention provides a recombinant Komagataella phaffii cell which is deficient for his4 and ura3, wherein said cell is transformed with:
(a) a first expression cassette which comprises a nucleic acid sequence encoding a recombinant protein fused to a nucleic acid sequence encoding the leader peptide according to SEQ ID No. 9 and operatively linked to a promoter according to SEQ ID No. 1, and a nucleic acid sequence according to SEQ ID No. 25 or a nucleic acid sequence which is 80% identical thereto encoding HIS4; and
(b) a second expression cassette which comprises a nucleic acid sequence encoding a recombinant protein fused to a nucleic acid sequence encoding the leader peptide according to SEQ ID No. 9 and operatively linked to a promoter according to SEQ ID No. 1, and a nucleic acid sequence according to SEQ ID No. 26 or a nucleic acid sequence which is 80% identical thereto encoding URA3.

The following examples are provided for illustrative purposes. It is thus understood that the examples are not to be construed as limiting. The skilled person will clearly be able to envisage further modifications of the principles laid out herein.

### EXAMPLES

### 1) Method for Pichia Expression Vector Construction and Screening

Gene parts were cloned into the pPICz or pAO815 expression vectors (Invitrogen). Expression vectors were linearized by restriction digest and the vector backbone was isolated using gel electrophoresis and purification. New DNA parts such as promoters, nucleic acid sequences encoding signal peptides, the recombinant protein, or the selection markers were cloned into the vector backbones by seamless ligation using the GeneArt Seamless cloning and assembly kit (Invitrogen) or by restriction ligation using T4 ligase (ThermoFischer).

The cloned constructs were transformed into an E. coli cloning host such as One Shot TOP10 (Invitrogen) or XL1-blue (Agilent), sequence verified, and then plasmid was purified. Purified expression vector plasmids were linearized by restriction digest and transformed into K. phaffi by electroporation. Transformants screened for transformation by zeomycin selection or by selection on minimal media. Individual colonies were screened first in microtiter plates for expression before growth in fermenters.

### 2) Fermentation Conditions

The expression vectors were transformed into Pichia pastoris (Komagataella phaffii) and the resulting strains were grown as described in the following.

For the fermentation condition with pulse feed of glucose, the initial batch of K. phaffii cells were seeded with a batch culture, with a pH that was greater than a pH of 4.0 and a temperature between 20-35 C.° The fermentation was sampled two times a day for 140 hours, with the first-time point taken at 24 hours after the time of inoculation. The initial batch was supplemented with 15 g/L of glucose (corn syrup). The cells were given a 1 g/L pulse of glucose after initial glucose was used up controlled by DO.

For the fermentation condition for Glucose limited, the initial batch of K. phaffii cells were seeded with a batch culture with a pH that was greater than a pH of 4.0 and a temperature between 20-35 C°. The fermentation was sampled two times a day for 140 hours, with the first-time point taken at 24 hours after the time of inoculation. The initial batch was supplemented with 15 g/L of glucose (corn syrup). After initial glucose was all used up, the cells were given a 1G/L pulse of glucose until the OD600 was more than 200, followed by constant feed of glucose.

The Pichia pastoris cultures transformed with expression vectors carrying the AOX promoter were treated with methanol according to procedures known in the art to induce gene expression.

### 3) Protein Quantitation

Lipase protein titers in the whole broth were measured by lipase activity by incubating the whole broth with p-octanoate as substrate in 50 mM HEPES pH 7.5 at a temperature of 26° C. for 10 minutes. Whole broth specific lipase activity was compared to a lipase gold standard which had been quantitated by amino acid analysis (AAA). Additionally, protein samples were subjected to SDS PAGE analysis.

The whole-broth titer obtained with different yeast strains is shown in Table 2:

| | | | Whole-broth | Fermentation |
|---|---|---|---|---|
| Enzyme | Promoter | Signal peptide | Titer (g/L) | Time (hr) |
| LIP62 | SEQ ID No. 8 | SEQ ID No. 14 | 3.8-5.4 | 146-168 |
| LIP62 | SEQ ID No. 8 | SEQ ID No. 15 | 0.9-8.8 | 141-189 |
| LIP117 | SEQ ID No. 8 | SEQ ID No. 15 | 1.7-2.5 | 118 |
| LIP120 | SEQ ID No. 8 | SEQ ID No. 15 | 1.1-11.8 | 118-214 |
| LIP62 | SEQ ID No. 8 | SEQ ID No. 10 | 2.8-9.0 | 124-145 |
| LIP134 | SEQ ID No. 8 | SEQ ID No. 10 | 3.5-13.7 | 145-189 |
| LIP160 | SEQ ID No. 8 | SEQ ID No. 15 | 1.8-2.7 | 118 |
| LIP157 | SEQ ID No. 8 | SEQ ID No. 15 | 1, 4 | 118 |
| LIP167 | SEQ ID No. 8 | SEQ ID No. 15 | 5.3-5.7 | 118 |
| LIP173 | SEQ ID No. 8 | SEQ ID No. 15 | 3.2-6.1 | 118-142 |
| LIP134 | SEQ ID No. 2 | SEQ ID No. 10 | 0.2-3.5 | 145-189 |
| LIP134 | SEQ ID No. 1 | SEQ ID No. 10 | 4.4-15.5 | 108-142 |
| LIP173 | SEQ ID No. 8 | SEQ ID No. 10 | 0.9-12.8 | 140-142 |
| LIP173 | SEQ ID No. 1 | SEQ ID No. 10 | 0.8-5.2 | 116-117 |
| LIP120 | SEQ ID No. 1 | SEQ ID No. 10 | 0.6-11.1 | 85-142 |
| LIP173 | SEQ ID No. 3 | SEQ ID No. 10 | 1.6-2.5 | 116-117 |
| LIP173 | SEQ ID No. 4 | SEQ ID No. 10 | 0.8-1.9 | 116-117 |
| LIP173 | SEQ ID No. 6 | SEQ ID No. 10 | 3.1-6.9 | 116-117 |
| LIP120 | SEQ ID No. 6 | SEQ ID No. 10 | 2.9-4.9 | 118 |

1. A recombinant yeast cell comprising at least a first expression cassette which comprises
(a) a promoter comprising a nucleic acid sequence which is at least 80% identical to the nucleic acid sequence according to any one of SEQ ID NOs. 1 to 8, operatively linked thereto
(b) a nucleic acid sequence encoding a leader peptide comprising an amino acid sequence which is at least 80% identical to the sequence according to any one of SEQ ID Nos. 9 to 15; and
(c) a nucleic acid sequence encoding a recombinant protein.

2. The recombinant yeast cell according to claim 1, further comprising a second expression cassette which comprises (a) a promoter comprising a nucleic acid sequence which is at least 80% identical to the sequence according to any one of SEQ ID NOs. 1 to 8, operatively linked thereto
(b) a nucleic acid sequence encoding a leader peptide comprising an amino acid sequence which is at least 80% identical to the sequence according to any one of SEQ ID NOs. 9 to 15; and
(c) a nucleic acid sequence encoding said recombinant protein.

3. The recombinant yeast cell according to claim 1, wherein the recombinant yeast cell is deficient for at least one marker gene and comprises a recombinant nucleic acid sequence encoding the marker gene.

4. The recombinant yeast cell according to claim 3, wherein the marker gene is an auxotrophic marker gene or an antibiotic resistance marker gene.

5. The recombinant yeast cell according to claim 4, wherein the auxotrophic marker gene is selected from the group consisting of ura3, his4, ade2, arg4, ade1, ura5, met2, lys2, pro3 and tyr1.

6. The recombinant yeast cell according to claim 4, wherein the antibiotic resistance marker gene is selected from the group consisting of zeocin resistance gene, kanamycin resistance gene, neomycin resistance gene, G418 resistance gene and hygromycin resistance gene.

7. The recombinant yeast cell according to claim 2, wherein the first and optionally the second expression cassette is stably integrated into genome of the recombinant yeast cell.

8. The recombinant yeast cell according to claim 1, wherein the recombinant yeast cell is deficient for a first marker gene and a second marker gene and wherein the recombinant yeast cell comprises a recombinant nucleic acid sequence encoding the first marker gene and a recombinant nucleic acid sequence encoding the second marker gene.

9. The recombinant yeast cell according to claim 8, wherein the first and the second marker gene is an antibiotic resistance marker gene independently selected from the group consisting of zeocin resistance gene, kanamycin resistance gene, neomycin resistance gene, G418 resistance gene and hygromycin resistance gene.

10. The recombinant yeast cell according to claim 8, wherein the first and the second marker gene is an auxotrophic marker gene independently selected from the group consisting of ura3, his4, ade2, arg4, ade1, ura5, met2, lys2, pro3 and tyr1.

11. The recombinant yeast cell according to claim 10, wherein the first marker gene is his4 and the second marker gene is ura3.

12. The recombinant yeast cell according to claim 11, comprising a deficiency of ura3, wherein the deficiency of ura3 is due to a deletion of part or the whole of a ura3 gene.

13. The recombinant yeast cell according to claim 10, wherein the nucleic acid sequence encoding the first auxotrophic marker is selected from the group consisting of:
(a) a nucleic acid sequence according to SEQ ID NO. 25;
(b) a nucleic acid sequence which is at least 65% identical to the nucleic acid sequence according to SEQ ID NO. 25;
(c) a nucleic acid sequence encoding the polypeptide according to SEQ ID NO. 27; and
(d) a nucleic acid sequence encoding a polypeptide which is at least 80% identical to the polypeptide according to SEQ ID NO. 27; and/or wherein the nucleic acid sequence encoding the second auxotrophic marker is selected from the group consisting of:
(i) a nucleic acid sequence according to SEQ ID NO. 26;
(ii) a sequence which is at least 65% identical to the nucleic acid sequence according to SEQ ID No. 26;
(iii) a nucleic acid sequence encoding the polypeptide according to SEQ ID NO. 28; and
(iv) a nucleic acid sequence encoding a polypeptide which is at least 80% identical to the polypeptide according to SEQ ID NO. 28.

14. The recombinant yeast cell according to claim 1, wherein the recombinant protein is an enzyme, a peptide, an antibody or antigen-binding fragment thereof, a protein antibiotic, a fusion protein, a vaccine or a vaccine-like protein or particle, a growth factor, a hormone or a cytokine.

15. The recombinant yeast cell according to claim 14, wherein the enzyme is a lipase, protease, alpha-amylase, beta-amylase, glucoamylase, xylanase, mannanase, glucanase, cellulase, or phytase.

16. The recombinant yeast cell according to claim 15, wherein the lipase is selected from:
(a) a lipase having an amino acid sequence according to SEQ ID NO. 23;
(b) a lipase having an amino acid sequence with at least 80% sequence identity to the amino acid sequence of SEQ ID NO. 23;
(c) a lipase having one or more amino acid substitutions on positions corresponding to positions 23, 33, 82, 83, 84, 85, 160, 199, 254, 255, 256, 258, 263, 264, 265, 268, 308 or 311 of SEQ ID NO. 23;
(d) a lipase encoded by a nucleic acid sequence according to SEQ ID No. 24; and
(e) a lipase encoded by any nucleic acid sequence encoding lipase with at least 80% sequence identity to the polypeptide sequence of SEQ ID NO. 23.

17. The recombinant yeast cell according to claim 1, wherein the yeast cell is a species of methylotropic yeast.

18. The recombinant yeast cell according to claim 1, wherein the yeast cell is a Komagataella phaffii cell.

19. A culture comprising the recombinant yeast cell according to claim 1.

20. A method for producing a recombinant protein, comprising the steps of:
(a) culturing the recombinant yeast cell according to claim 1 in a culture medium; and
(b) obtaining the recombinant protein.

21. The recombinant yeast cell of claim 1, wherein the first expression cassette comprises the promoter sequence of SEQ ID NO. 1 operatively linked to the leader peptide sequence of SEQ ID NO. 10.

22. The recombinant yeast cell of claim 2, wherein the second expression cassette comprises the promoter sequence of SEQ ID NO. 1 operatively linked to the leader peptide sequence of SEQ ID NO. 10.
